(19) United States
(12) Patent Application Publication
(10) Pub. No.: US 2016/0160297 A1
(43) Pub. Date: Jun. 9, 2016

(54) METHODS AND COMPOSITIONS FOR DETECTING MUTATIONS IN THE HUMAN PI3KCA (PIK3CA) GENE

(71) Applicant: Roche Molecular Systems, Inc., Pleasanton, CA (US)

(72) Inventor: Alison Tsan, Danville, CA (US)

(21) Appl. No.: 15/017,179

(22) Filed: Feb. 5, 2016

Related U.S. Application Data
(62) Division of application No. 14/205,751, filed on Mar. 12, 2014.
(60) Provisional application No. 61/780,017, filed on Mar. 13, 2013.

(51) Int. Cl. C12Q 1/68

(52) U.S. Cl. CPC C12Q 1/6886; C12Q 2600/156

(57) ABSTRACT

The invention comprises reagents and methods for detecting cancer-associated mutations in the human PI3KCA (PIK3CA) gene and assessing the patients based thereon.

### CROSS-REFERENCE TO RELATED APPLICATIONS

The present application is a divisional of U.S. Ser. No. 14/205,751, filed Mar. 12, 2014, which claims priority to U.S. Ser. No. 61/780,017, filed Mar. 13, 2013, the disclosures of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Feb. 4, 2014, is named 31384-USI_SL.txt and is 58,772 bytes in size.

### FIELD OF THE INVENTION

The invention relates to cancer diagnostics and companion diagnostics for cancer therapies. In particular, the invention relates to methods and compositions for detection of mutations that are useful for diagnosis and prognosis as well as predicting the effectiveness of treatment of cancer.

### BACKGROUND OF THE INVENTION

Phosphatidylinositol 3-kinases (PI3Ks) are intracellular lipid kinases that regulate signaling pathways controlling cell proliferation and survival, adhesion and motility. (Vivanco and Sawyers, (2002) The phosphatidylinositol 3-Kinase AKT pathway in human cancer, Nature Rev. Cancer 2:489). PI3KCA (PIK3CA) is a member of the PI3K gene family encoding the catalytic subunit of the kinase p110α. This gene is of unique relevance for neoplasia: of all the PI3K genes tested, only PI3KCA was found mutated in multiple cancers. In one study, somatic mutations in the PI3KCA gene were found in 32% of colon cancers, 27% glioblastomas, 25% gastric cancers, 8% breast cancers and 4% lung cancers. (Samuels et al. (2004) High frequency of mutations in the PI3KCA gene in human cancers, Science 304:554.) Later studies reported mutations also in uterine (24%), ovarian (10%) and cervical (10%) cancer Brana and Sui (2012) Clinical development of phosphatidylinositol 3-kinase inhibitors for cancer treatment. BMC Medicine 2012, 10:161.

PI3K activates the intracellular Akt/mTOR pathway by specifically activating the Akt protein. A genetic approach revealed that constitutive activation of this pathway by the mutant PI3KCA contributes to resistance to EGFR targeting therapies. (Berns et al. (2007) A functional genetic approach identifies the PI3K pathway as a major determinant of trastuzumab resistance in breast cancer, Cancer Cell 12:395. At the same time, it was demonstrated that an intact (non-mutated) PI3KCA activity may be suppressed by specific inhibitors thus overcoming the effect of the disregulated upstream element in the pathway (e.g. EGFR) and recently, therapeutic agents targeting PI3KCA (p110α) itself have been developed (reviewed in Weickhardt et al. (2010) Strategies for Overcoming Inherent and Acquired Resistance to EGFR Inhibitors by Targeting Downstream Effectors in the RAS/PI3K Pathway, Current Cancer Drug Targets, 10:824; and Brana and Sui (2012) Clinical development of phosphatidylinositol 3-kinase inhibitors for cancer treatment, BMC Medicine 2012, 10:161.

Taken together, these studies demonstrate the need for methods and tools for detecting somatic mutations in the PI3KCA gene for delivering personalized healthcare to patients seeking targeted cancer therapies.

To date, over 30 somatic mutations in the PI3KCA gene have been identified. (U.S. Pat. No. 8,026,053.) The majority of the mutations cluster in exons 9 and 20. However a number of clinically significant mutations have been reported in exons 1, 4 and 7 as well. A diagnostic assay should target as many of these mutations as possible. Furthermore, precise discrimination (high specificity) is required since the output of the assay will determine the course of a patient's cancer therapy.

### SUMMARY OF THE INVENTION

The invention comprises oligonucleotides for detecting each of the mutations H1047L, H1047R, H1047Y, N345K, E542K, E545A, E545G, E545K, G1049R, M1043I, Q546E, Q546L and Q546K in the human PIK3CA gene, that are at least 90% identical to and have the 3′-terminal nucleotide of one of the following: SEQ ID NOs: 2, 18, 39, 61, 84, 100, 127, 148, 170, 185, 197, 208 and 219.

In other embodiments, the invention is a method of assaying a sample for the presence of one or more mutations H1047L, H1047R, H1047Y, N345K, E542K, E545A, E545G, E545K, G1049R, M1043I, Q546E, Q546L and Q546K in the human PIK3CA gene comprising contacting the sample with an allele-specific oligonucleotide for each mutation, wherein the oligonucleotide shares at least 90% identity with and has the same 3-terminal nucleotide as an oligonucleotide selected from a group consisting of SEQ ID NOs: 2, 18, 39, 61, 84, 100, 127, 148, 170, 185, 197, 208, 219 and comprises at least one mismatch with the naturally-occurring sequence of the human PIK3CA gene among the penultimate 5 nucleotides at the 3′-terminus of the oligonucleotide. In variations of this embodiment, the allele-specific oligonucleotide is selected from a group consisting of SEQ ID NOs: 8, 21, 46, 78, 93, 113, 141, 166, 170, 194, 199, 217 and 228. The allele-specific oligonucleotide may comprise at least one nucleotide with a modified base.

In yet other embodiments, the invention is a set of oligonucleotides for detecting one or more mutations H1047L, H1047R, H1047Y, N345K, E542K, E545A, E545G, E545K, G1049R, M1043I, Q546E, Q546L and Q546K mutations in the PIK3CA gene comprising a combination of two or more oligonucleotides sharing at least 90% identity with and having the same 3-terminal nucleotide as: SEQ ID NOs: 2, 18, 39, 61, 84, 100, 127, 148, 170, 185, 197, 208 and 219 and comprising at least one mismatch with the naturally-occurring sequence of the human PIK3CA gene among the penultimate 5 nucleotides at the 3′-terminus of the oligonucleotide. In variations of this embodiment, the oligonucleotides are selected from SEQ ID NOs: 8, 21, 46, 78, 93, 113, 141, 166, 170, 194, 199, 217 and 228. The oligonucleotides may also comprise at least one nucleotide with a modified base

In yet other embodiments, the invention is a reaction mixture for detecting one or more mutations H1047L, H1047R, H1047Y, N345K, E542K, E545A, E545G, E545K, G1049R, M1043I, Q546E, Q546L and Q546K in the human PIK3CA gene comprising one allele-specific oligonucleotide for each mutation sharing at least 90% identity with and having the same 3-terminal nucleotide as an oligonucleotide selected from a group consisting of SEQ ID NOs: 2, 18, 39, 61, 84, 100, 127, 148, 170, 185, 197, 208, 219 and comprising at least one mismatch with the naturally-occurring sequence of the human PIK3CA gene among the penultimate 5 nucleotides at the 3′-terminus of the oligonucleotide. In variations of this embodiment, the mixture comprises a combination of two or more of: SEQ ID NOs: 8, 21, 46, 78, 93, 113, 141, 166, 170, 194, 199, 217 and 228. The two or more oligonucleotides may comprise at least one nucleotide with a modified base.

In yet other embodiments, the invention is a method of assessing cancer in a patient by detecting in the patient's sample one or more of the mutations H1047L, H1047R, H1047Y, N345K, E542K, E545A, E545G, E545K, G1049R, M1043I, Q546E, Q546L and Q546K in the human PIK3CA gene comprising contacting the sample with one allele-specific nucleotide oligonucldeotide for each mutation sharing at least 90% identity with and having the same 3-terminal nucleotide as an oligonucleotide selected from a group consisting of SEQ ID NOs: 2, 18, 39, 61, 84, 100, 127, 148, 170, 185, 197, 208, 219 and comprising at least one mismatch with the naturally-occurring sequence of the human PIK3CA gene among the penultimate 5 nucleotides at the 3′-terminus of the oligonucleotide. In variations of this embodiment, the allele-specific oligonucleotide is selected from a group consisting of SEQ ID NOs: 8, 21, 46, 78, 93, 113, 141, 166, 170, 194, 199, 217 and 228. The allele-specific oligonucleotide may comprise at least one nucleotide with a modified base.

### DETAILED DESCRIPTION OF THE INVENTION

### Definitions

To facilitate the understanding of this disclosure, the following definitions of the terms used herein are provided.

The term “X[n]Y” refers to a missense mutation that results in a substitution of amino acid X for amino acid Y at position [n] within the amino acid sequence. For example, the term “H1047R” refers to a mutation where histidine at position 1047 is replaced with arginine.

The term “allele-specific primer” or “AS primer” refers to a primer that hybridizes to more than one variant of the target sequence, but is capable of discriminating between the variants of the target sequence in that only with one of the variants, the primer is efficiently extended by the nucleic acid polymerase under suitable conditions. With other variants of the target sequence, the extension is less efficient or inefficient.

The term “common primer” refers to the second primer in the pair of primers that includes an allele-specific primer. The common primer is not allele-specific, i.e. does not discriminate between the variants of the target sequence between which the allele-specific primer discriminates.

The term “assessing” in connection with cancer refers to inferring the status or condition of the cancer as well as determining the need for diagnostic procedures or treatments, evaluating potential effectiveness of the treatments, monitoring the subject's cancer, or any other steps or processes related to treatment or diagnosis of a cancer.

The terms “complementary” or “complementarity” are used in reference to antiparallel strands of polynucleotides related by the Watson-Crick base-pairing rules. The terms “perfectly complementary” or “100% complementary” refer to complementary sequences that have Watson-Crick pairing of all the bases between the antiparallel strands, i.e. there are no mismatches between any two bases in the polynucleotide duplex. However, duplexes are formed between antiparallel strands even in the absence of perfect complementarity. The terms “partially complementary” or “incompletely complementary” refer to any alignment of bases between antiparallel polynucleotide strands that is less than 100% perfect (e.g., there exists at least one mismatch or unmatched base in the polynucleotide duplex). The duplexes between partially complementary strands are generally less stable than the duplexes between perfectly complementary strands.

The term “sample” refers to any composition containing or presumed to contain nucleic acid. This includes a sample of tissue or fluid isolated from an individual for example, skin, plasma, serum, spinal fluid, lymph fluid, synovial fluid, urine, tears, blood cells, organs and tumors, and also to samples of in vitro cultures established from cells taken from an individual, including the formalin-fixed paraffin embedded tissues (FFPET) and nucleic acids isolated therefrom. To detect a somatic mutation, the sample is typically comprises a fragment of a solid tumor (primary or metastatic) or tumor-derived cells found elsewhere in the body, e.g. in circulating blood.

The terms “polynucleotide” and “oligonucleotide” are used interchangeably. “Oligonucleotide” is a term sometimes used to describe a shorter polynucleotide. An oligonucleotide may be comprised of at least 6 nucleotides, for example at least about 10-12 nucleotides, or at least about 15-30 nucleotides corresponding to a region of the designated nucleotide sequence.

The term “primary sequence” refers to the sequence of nucleotides in a polynucleotide or oligonucleotide. Nucleotide modifications such as nitrogenous base modifications, sugar modifications or other backbone modifications are not a part of the primary sequence. Labels, such as chromophores conjugated to the oligonucleotides are also not a part of the primary sequence. Thus two oligonucleotides can share the same primary sequence but differ with respect to the modifications and labels.

The term “primer” refers to an oligonucleotide which hybridizes with a sequence in the target nucleic acid and is capable of acting as a point of initiation of synthesis along a complementary strand of nucleic acid under conditions suitable for such synthesis. As used herein, the term “probe” refers to an oligonucleotide which hybridizes with a sequence in the target nucleic acid and is usually detectably labeled. The probe can have modifications, such as a 3′-terminus modification that makes the probe non-extendable by nucleic acid polymerases, and one or more chromophores. An oligonucleotide with the same sequence may serve as a primer in one assay and a probe in a different assay.

The term “modified nucleotide” refers to a unit in a nucleic acid polymer that contains a modified base, sugar or phosphate group, or that incorporates a non-natural moiety in its structure. Examples of non-natural nucleotides, include nucleotides with a modified nitrogenous base, e.g. alkylated or otherwise substitutes with a group not present among the conventional nitrogenous bases involved in Watson-Crick pairing. By way of illustration and not limitation, modified nucleotides include those with bases substituted with methyl, ethyl, benzyl or butyl-benzyl groups.

As used herein, the term “target sequence”, “target nucleic acid” or “target” refers to a portion of the nucleic acid sequence which is to be either amplified, detected or both.

The terms “hybridized” and “hybridization” refer to the base-pairing interactions between two nucleic acids that result in formation of a duplex. It is not a requirement that two nucleic acids have 100% complementarity over their full length to achieve hybridization.

The present invention comprises methods and compositions for rapid and precise determination of the presence of one or more of the mutations in the PI3KCA gene in patient's samples. The invention enables detection of the mutations selected from H1047L, H1047R, H1047Y, N345K, E542K, E545A, E545G, E545K, G1049R, M1043I, Q546E, Q546L and Q546K as well as a simultaneous query for 1, 2, 3, 4, 5, 6, 7, 8, 9, 10, 11, 12 or 13 of the mutations listed above.

One technique that is sensitive and amenable to multiplexing is allele-specific PCR (AS-PCR) described in e.g. U.S. Pat. No. 6,627,402. This technique detects mutations or polymorphisms in nucleic acid sequences in the presence of wild-type variants of the sequences. In a successful allele-specific PCR, the desired variant of the target nucleic acid is amplified, while the other variants are not, at least not to a detectable level.

One measure of discrimination of an allele-specific PCR is the difference between Cₜ values (ΔCₜ) in the amplification reactions involving the two alleles. Each amplification reaction is characterized by a “growth curve” or “amplification curve” in the context of a nucleic acid amplification assay is a graph of a function, where an independent variable is the number of amplification cycles and a dependent variable is an amplification-dependent measurable parameter measured at each cycle of amplification, such as fluorescence emitted by a fluorophore. Typically, the amplification-dependent measurable parameter is the amount of fluorescence emitted by the probe upon hybridization, or upon the hydrolysis of the probe by the nuclease activity of the nucleic acid polymerase, see Holland et al., (1991) Proc. Natl. Acad. Sci. 88:7276-7280 and U.S. Pat. No. 5,210,015. A growth curve is characterized by a “threshold value” (or Cₜ value) which is a number of cycles where a predetermined magnitude of the measurable parameter is achieved. A lower Cₜ value represents more rapid amplification, while the higher Cₜ value represents slower amplification. In the context of an allele-specific reaction the difference between Cₜ values of the two templates represents allelic discrimination in the reaction.

In an allele-specific PCR, at least one primer is allele-specific such that primer extension occurs only (or preferentially) when the specific variant of the sequence is present and does not occur (or occurs less efficiently, i.e. with a substantial ΔCₜ) when another variant is present. Design of successful allele-specific primers is an unpredictable art. While it is routine to design a primer for a known sequence, no formula exists for designing a primer that can discriminate between very similar sequences. The discrimination is especially challenging when one or more allele-specific primers targeting one or more polymorphic sites are present in the same reaction mixture.

Typically, the discriminating nucleotide in the primer, i.e. the nucleotide matching only one variant of the target sequence, is the 3′-terminal nucleotide. However, the 3′ terminus of the primer is only one of many determinants of specificity. For example, additional mismatches may also affect discrimination. See U.S. patent application Ser. No. 12/582,068 filed on Oct. 20, 2009 (published as US20100099110.) Another approach is to include non-natural or modified nucleotides that alter base pairing between the primer and the target sequence (U.S. Pat. No. 6,001,611, incorporated herein in its entirety by reference.) The reduced extension kinetics and thus specificity of a primer is influenced by many factors including overall sequence context of the mismatch and other nucleic acids present in the reaction. The effect of these external factors on each additional mismatch as well as of each additional non-natural nucleotide either alone or in combination cannot be predicted. The applicants tested multiple variants of the primers and found that surprisingly, certain variants are dramatically different with respect to their ability to discriminate between closely related target sequences.

For successful extension of a primer, complementarity at the 3′-end of the primer is more critical than complementarity at the 5′-end of the primer. (Innis et al. Eds. PCR Protocols, (1990) Academic Press, Chapter 1, pp. 9-11). Therefore the present invention encompasses the primers disclosed in Tables 1-13 as well as equivalents thereof with 5′-end variations.

In one embodiment the present invention comprises oligonucleotides for detecting PI3KCA mutations selected from H1047L, H1047R, H1047Y, N345K, E542K, E545A, E545G, E545K, G1049R, M1043I, Q546E, Q546L and Q546K as well as a simultaneous query for 1, 2, 3, 4, 5, 6, 7, 8, 9, 10, 11, 12 or 13 of the mutations listed above. In one embodiment, the invention comprises oligonucleotides selected from SEQ ID NOs: 2, 18, 39, 61, 84, 100, 127, 148, 170, 185, 197, 208 and 219 (Tables 1-13) as well as variations at least 90% identical to and having the 3′-terminal nucleotide of said oligonucleotides, for specifically detecting mutations in the human PI3KCA gene. As illustrated in Tables 1-13, oligonucleotides sharing 90% identity with a given oligonucleotide include those having 1, 2 or 3 mismatches with that oligonucleotide. As further illustrated in Tables 1-13, oligonucleotides sharing 90% identity with a given oligonucleotide also include those having one or more non-natural nucleotide. As further illustrated in Tables 1-13, the mismatches and non-natural nucleotides typically occur within the 3′-terminal portion of the oligonucleotide, specifically within 5 penultimate nucleotides. However, some oligonucleotides sharing 90% identity with a given oligonucleotide also include those having 1, 2 or 3 mismatches elsewhere in the oligonucleotide, e.g. in the 5′-portion of the oligonucleotide. As demonstrated in examples below, the oligonucleotides of the present invention are characterized by a substantial positive ΔCₜ determined using the formula ΔCₜ=Cₜ(wild type)−Cₜ(mutant), indicating that amplification of the wild-type template is detectably slower than that of the mutant template.

Legends to the Tables

The underlined nucleotides are mismatched with both the wild-type and the mutant sequence. The following abbreviations are used for the modified-base nucleotides: A* and C* are respectively N6-tert-butyl-benzyl-deoxyadenine and N4-tert-butyl-benzyl-deoxycytosine, Ĉ is N4-ethyl-deoxycytosine; and C^{#} is N4-methyl-deoxycytosine.

TABLE 1
Oligonucleotides for
detecting mutation H1047L

| SEQ ID NO: | SEQUENCE 5′-3′ |
|---|---|
| 1 | TTTTGTTGTCCAGCCACCATGAT |
| 2 | TTTTGTTGTCCAGCCACCATGAA |
| 3 | TTTTGTTGTCCAGCCACCATGCA |
| 4 | TTTTGTTGTCCAGCCACCATGGA |
| 5 | TTTTGTEGTCCAGCCACCATGTA |
| 6 | TTTTGTTGTCCAGCCACCATCAA |
| 7 | TTTTGTTGTCCAGCCACCATTAA |
| 8 | TTTTGTTGTCCAGCCACCATAAA |
| 9 | TTTTGTTGTCCAGCCACCAAGAA |
| 10 | TTTTGTTGTCCAGCCACCACGAA |
| 11 | TTTTGTTGTCCAGCCACCAGGAA |
| 12 | GTFTTTTGTTGTCCAGCCACCATGAA |
| 13 | GTTTTTGTTGTCCAGCCACCATGAA* |
| 14 | GTTTTTGTTGTCCAGCCACCATGA*A |
| 15 | CC*GTTTTTGTTGTC*CAGC*CACC*ATGA*A |
| 16 | AATCC*ATTGTTGTTGTC*CAGC*CACC*ATGAA* |

TABLE 2
Oligomicleotides for
detecting mutation H1047R

| SEQ ID NO: | SEQUENCE 5′-3′ |
|---|---|
| 17 | TTTGTTGTCCAGCCACCATGAT |
| 18 | TTTGTTGTCCAGCCACCATGCC |
| 19 | TTTGTTGTCCAGCCACCATGGC |
| 20 | TTTGTTGTCCAGCCACCATGTC |
| 21 | TTTGTTGTCCAGCCACCATCAC |
| 22 | TTTGTTGTCCAGCCACCATTAC |
| 23 | TTTGTTGTCCAGCCACCATAAC |
| 24 | TTTGTTGTCCAGCCACCAAGAC |
| 25 | TTTGTEGTCCAGCCACCACGAC |
| 26 | TTTGTTGTCCAGCCACCAGGAC |
| 27 | TTTGTTGTCCAGCCACCATGAT |
| 2$ | TTTGTTGTCCAGCCACCATGCC |
| 29 | TTTCATGAAACAAATGAATGATGCAGG |
| 30 | TTTCATGAAACAAATGAATGATGCATG |
| 31 | TTTCATGAAACAAATGAATGATGCAAG |
| 32 | TTTCATGAAACAAATGAATGATGCCCG |
| 33 | TTTCATGAAACAAATGAATGATGCGCG |
| 34 | TTTCATGAAACAAATGAATGATGCTCG |
| 35 | TTTCATGAAACAAATGAATGATGGACG |
| 36 | TTTCATGAAACAAATGAATGATGTACG |
| 37 | TTTCATGAAACAAATGAATGATGAACG |

TABLE 3
Oligonucleotides for
detecting mutation H1047Y

| SEQ ID NO: | SEQUENCE 5′-3′ |
|---|---|
| 38 | TTTGTTGTCCAGCCACCATGATG |
| 39 | TTTGTTGTCCAGCCACCATGAAA |
| 40 | TTTGTTGTCCAGCCACCATGACA |
| 41 | TTTGTTGTCCAGCCACCATGAGA |
| 42 | TTTGTTGTCCAGCCACCATGCTA |
| 43 | TTTGTTGTCCAGCCACCATGGTA |
| 44 | TTTGTTGTCCAGCCACCATGTTA |
| 45 | TTTGTTGTCCAGCCACCATCATA |
| 46 | TTTGTTGTCCAGCCACCATTATA |
| 47 | TTTGTTGTCCAGCCACCATAATA |
| 48 | GTTTTGTTGTCCAGCCACCATGA*TA |
| 49 | TTGTGTTGTCCAGCCACCATGA*TA |
| 50 | AGTATTTCATGAAACAAATGAATGATGCGT |
| 51 | AGTATTTCATGAAACAAATGAATGATGCTT |
| 52 | AGTATTTCATGAAACAAATGAATGATGGAT |
| 53 | AGTATTTCATGAAACAAATGAATGATGTAT |
| 54 | AGTATTTCATGAAACAAATGAATGATGAAT |
| 55 | AGTGTTTCATGAAACAAATGAATGATGCA*T |
| 56 | AGTGTTTCATGAAACAAATGAATGATGC*AT |
| 57 | AGTATTTCATGAAACAAATGAATGATGC^GT |
| 58 | AGTATTTCATGAAACAAATGAATGATTCA*T |
| 59 | AGTATTTCATGAAACAAATGAATGATGC^TT |

TABLE 4
Oligonucleotides for
detecting mutation N345K

| SEQ ID NO: | SEQUENCE 5′-3′ |
|---|---|
| 60 | ATAAAAATTCTTTGTGCAACCTACGTGAAT |
| 61 | ATAAAAATTCTTTGTGCAACCTACGTGAAA |
| 62 | ATAAAAATTCTTTGTGCAACCTACGTGACA |
| 63 | ATAAAAATTCTTTGTGCAACCTACGTGAGA |
| 64 | ATAAAAATTCTTTGTGCAACCTACGTGATA |
| 65 | ATAAAAATTCTTTGTGCAACCTACGTGCAA |
| 66 | ATAAAAATTCTTTGTGCAACCTACGTGGAA |
| 67 | ATAAAAATTCTTTGTGCAACCTACGTGTAA |
| 68 | ATAAAAATTCTTTGTGCAACCTACGTCAAA |
| 69 | ATAAAAATTCTTTGTGCAACCTACGTTAAA |
| 70 | ATAAAAATTCTTTGTGCAACCTACGTAAAA |
| 71 | ATAGAAATTCTTTGTGCAACCTACGTGAAA |
| 72 | ATGAAAATTCTTTGTGCAACCTACGTGAAA* |
| 73 | ATGAAAATTCTTTGTGCAACCTACGTGAA*A |
| 74 | ATGAAAATTCTTTGTGCAACCTACGTGA*AA |
| 75 | ATAAAAATTCTTTGTGCAACCTACGTGAC*A |
| 76 | ATAAAAATTCTTTGTGCAACCTACGTGC*AA |
| 77 | ATAAAAATTCTTTGTGCAACCTACGTGAC#A |
| 78 | ATAAAAATTCTTTGTGCAAGCTACGGGAAA* |
| 79 | ATAAAAATTCTTTGTGCAACCTACGTC*AAA |
| 80 | ATAAAAATTCTTTGTGCAACCTACGGGAA*A |
| 81 | ATAAAAATTCTTTGTGCAACCTACGTC#AAA |
| 82 | ATAAAAATTCTTTGTGCAACCTACGTC*AAA* |

TABLE 5
Oligonucleotides for
detecting mutation E542K

| SEQ ID NO: | SEQUENCE 5′-3′ |
|---|---|
| 83 | CAATTTCTACACGAGATCCTCTCTCTG |
| 84 | CAATTTCTACACGAGATCCTCTCTCTA |
| 85 | CAATTTCTACACGAGATCCTCTCTCAA |
| 86 | CAATTTCTACACGAGATCCTCTCTCCA |
| 87 | CAATTTCTACACGAGATCCTCTCTCGA |
| 88 | CAATTTCTACACGAGATCCTCTCTGTA |
| 89 | CAATTTCTACACGAGATCCTCTCTTTA |
| 90 | CAATTTCTACACGAGATCCTCTCTATA |
| 91 | CAATTTCTACACGAGATCCTCTCACTA |
| 92 | CAATTTCTACACGAGATCCTCTCCCTA |
| 93 | CAATTTCTACACGAGATCCTCTCGCTA |
| 94 | CAGTTTCTACACGAGATCCTCTCTCTA |
| 95 | GAAGCAATTTCTACACGAGATCCTCTCTCTA* |
| 96 | GAAGCAATTTCTACACGAGATCCTCTCTC*TA |
| 97 | CAGTTTCTACACGAGATCCTCTCTC*TA |

TABLE 6
Oilgonucleotides for
detecting mutation E545A

| SEQ ID NO: | SEQUENCE 5′-3′ |
|---|---|
| 98 | GAGATCCTCTCTCTGAAATCACTGA |
| 99 | GAGATCCTCTCTCTGAAATCACTGC |
| 100 | GAGATCCTCTCTCTGAAATCACTCC |
| 101 | GAGATCCTCTCTCTGAAATCACTTC |
| 102 | GAGATCCTCTCTCTGAAATCACTAC |
| 103 | GAGATCCTCTCTCTGAAATCACAGC |
| 104 | GAGATCCTCTCTCTGAAATCACCGC |
| 105 | GAGATCCTCTCTCTGAAATCACGGC |
| 106 | GAGATCCTCTCTCTGAAATCAGTGC |
| 107 | GAGATCCTCTCTCTGAAATCATTGC |
| 108 | GAGATCCTCTCTCTGAAATCAATGC |
| 109 | GGGATCCTCTCTCTGAAATCACTGC |
| 110 | GGGATCCTCTCTCTGAAATCAC*TGC |
| 111 | GGGATCCTCTCTCTGAAATCACTGC* |
| 112 | GAGATCCTCTCTCTGAAATCGCTGC* |
| 113 | GAGATCCTCTCTCTGAAATCATTGC* |
| 114 | GAGATCCTCTCTCTGAAATCACTC*C |
| 115 | GAGATCCTCTCTCTGAAATCA*CTLC |
| 116 | GAGATCCTCTCTCTGAAATCACC*GC |
| 117 | GAGATCCTCTCTCTGAAATCACTA*C |
| 118 | GAGATCCTCTCTCTGAAATCGCC^GC |
| 119 | GAGATCCTCTCTCTGAAATCACCGC* |
| 120 | GAGATCCTCTCTCTGAAATCACGGC* |
| 121 | GAGATCCTCTCTCTGAAATCAC^TC^C |
| 122 | GAGATCCTCTCTCTGAAATCAC^GGC |
| 123 | GAGATCCTCTCTCTGAAATCAC*CGC |
| 124 | GAGATCCTCTCTCTGAAATCACA*GC |

L-Gciamp

TABLE 7
Oligonucleotides for
detecting rotation E545G

| SEQ ID NO: | SEQUENCE 5′-3′ |
|---|---|
| 125 | GAGATCCTCTCTCTGAAATCACTGA |
| 126 | GAGATCCTCTCTCTGAAATCACTGG |
| l27 | GAGATCCTCTCTCTGAAATCACTCG |
| 128 | GAGATCCTCTCTCTGAAATCACTAG |
| 129 | GAGATCCTCTCTCTGAAATCACTTG |
| 130 | GAGATCCTCTCTCTGAAATCACAGG |
| 131 | GAGATCCTCTCTCTGAAATCACCGG |
| 132 | GAGATGCTCTCTCTGAAATCACGGG |
| 133 | GAGATCCTCTCTCTGAAATCAGTGG |
| 134 | GAGATCCTCTCTCTGAAATCAATGG |
| 135 | GAGATCCTCTCTCTGAAATCATTGG |
| 136 | GGGATCCTCTCTCTGAAATCACTGG |
| 137 | GGGATCCTCTCTCTGAAATCAC*TGG |
| 138 | GAGATCCTCTCTCTGAAATCACTC*G |
| 139 | GAGATCCTCTCTCTGAAATCA*CTC\hat{ }G |
| 140 | GAGATCCTCTCTCTGAAATCA*CTTG |
| 141 | GAGATCCTCTCTCTGAAATCACTA*G |
| 142 | GAGATCCTCTCTCTGAAATCACTC\hat{ }G |
| 113 | GAGATCCTCTCTCTGAAATCAA*TGG |
| 144 | CTATACGAGATCCTCTCTCTIAAATCAC*TGG |
| 145 | AGATCCTCTCTCTGAAATCACTAG |
| 146 | AGATCCTCTCTCTGAAATCACGGG |

TABLE 8
Oligonucleotides for
detecting mutation E545K

| SEQ ID NO: | SEQUENCE 5′-3′ |
|---|---|
| 147 | ACGAGATCCTCTCTCTGAAATCACTG |
| 148 | ACGAGATCCTCTCTCTGAAATCACTA |
| 149 | ACGAGATCCTCTCTCTGAAATCACAA |
| 150 | ACGAGATCCTCTCTCTGAAATCACCA |
| 151 | ACGAGATCCTCTCTCTGAAATCACGA |
| 152 | ACGAGATCCTCTCTCTGAAATCAGTA |
| 153 | ACGAGATCCTCTCTCTGAAATCAATA |
| 154 | ACGAGATCCTCTCTCTGAAATCATAA |
| 155 | ACGAGATCCTCTCTCTGAAATCCCTA |
| 156 | ACGAGATCCTCTCTCTGAAATCGCTA |
| 157 | ACGAGATCCTCTCTCTGAAATCTCTA |
| 158 | AGGAGATCCTCTCTCTGAAATCACTA |
| 159 | AGGAGATCCTCTCTCTGAAATCACTA* |
| 160 | AGGAGATCCTCTCTCTGAAATCACATA |
| 161 | AGGAGATCCTCTCTCTGAAATCA*CTA |
| 162 | ACGAGATCCTCTCTCTGAAATCAA*TA |
| 163 | ACGAGATCCTCTCTCTGAAATCACA*A |
| 164 | ACGAGATCCTCTCTCTGAAATC#AA*TA |
| 165 | ACGAGATCCTCTCTCTGAAATCACC*A |
| 166 | ACGAGATCCTCTCTCTGAAATCC*CTA |

TABLE 9
Oligonudeotides for
detecting mutation Q546K

| SEQ ID NO: | SEQUENCE 5′-3′ |
|---|---|
| 167 | AGATCCTCTCTCTGAAATCACTGAGC |
| 168 | AGATCCTCTCTCTGAAATCACTGAGA |
| 169 | AGATCCTCTCTCTGAAATCACTGACA |
| 170 | AGATCCTCTCTCTGAAATCACTGAAA |
| 171 | AGATCCTCTCTCTGAAATCACTGATA |
| 172 | AGATCCTCTCTCTGAAATCACTGCGA |
| 173 | AGATCCTCTCTCTGAAATCACTGTGA |
| 174 | AGATCCTCTCTCTGAAATCACTGGGA |
| 175 | AGATCCTCTCTCTGAAATCACTCAGA |
| 176 | AGATCCTCTCTCTGAAATCACTAAGA |
| 177 | AGATCCTCTCECTGAAATCACTTAGA |
| 178 | AGGTCCTCTCTCTGAAATCACTGAGA |
| 179 | GAGGTCCTCTCTCTGAAATCACTGAGA* |
| 180 | GAGGTCCTCTCTCTGAAATCACTGA*GA |
| 181 | GAGATCCTCTCTCTGAAATCACTGAAA |
| 182 | GAGATCCTCTCTCTGAAATCACTGGGA |
| 183 | GAGATCCTCTCTCTGAAATCACTAAGA |

TABLE 10
Oligonucleotides for
detecting mutation Q546E

| SEQ ID NO: | SEQUENCE 5′-3′ |
|---|---|
| 184 | ATCCTCTCTCTGAAATCACTGAGC |
| 185 | ATCCTCTCTCTGAAATCACTGAGG |
| 186 | ATCCTCTCTCTGAAATCACTGAAG |
| 187 | ATCCTCTCTCTGAAATCACTGACG |
| 188 | ATCCTCTCTCTGAAATCACTGATG |
| 189 | ATCCTCTCTCTGAAATCACTGCGG |
| 190 | ATCCTCTCTCTGAAATCACTGGGG |
| 191 | ATCCTCTCTCTGAAATCACTGTGG |
| 192 | ATCCTCTCTCTGAAATCACTAAGG |
| 193 | ATCCTCTCTCTGAAATCACTCAGG |
| 194 | ATCCTCTCTCTGAAATCACTTAGG |

TABLE 11
Oligonucleotides for
detecting mutation Q546L

| SEQ ID NO: | SEQUENCE 5′-3′ |
|---|---|
| 195 | TCCTCTCTCTGAAATCACTGAGCA |
| 197 | TCCTCTCTCTGAAATCACTGAGCT |
| 198 | TCCTCTCTCTGAAATCACTGAGAT |
| 199 | TCCTCTCTCTGAAATCACTGAGGT |
| 200 | TCCTCTCTCTGAAATCACTGAGTT |
| 201 | TCCTCTCTCTGAAATCACTGAACT |
| 202 | TCCTCTCTCTGAAATCACTGACCT |
| 203 | TCCTCTCTCTGAAATCACTGATCT |
| 204 | TCCTCTCTCTGAAATCACTGCGCT |
| 205 | TCCTCTCTCTGAAATCACTGGGCT |
| 206 | TCCTCTCTCTGAAATCACTGTGCT |

TABLE 12
Oligonucleotides for
detecting mutation G1049R

| SEQ ID NO: | SEQUENCE 5′-3′ |
|---|---|
| 207 | CATGAAACAAATGAATGATGCACATCATG |
| 208 | CATGAAACAAATGAATGATGCACATCATC |
| 209 | CATGAAACAAATGAATGATGCACATCAAC |
| 210 | CATGAAACAAATGAATGATGCACATCACC |
| 211 | CATGAAACAAATGAATGATGCACATCAGC |
| 212 | CATGAAACAAATGAATGATGCACATCCTC |
| 213 | CATGAAACAAATGAATGATGCACATCGTC |
| 214 | CATGAAACAAATGAATGATGCACATGTTC |
| 215 | CATGAAACAAATGAATGATGCACATAATC |
| 216 | CATGAAACAAATGAATGATGCACATGATC |
| 217 | CATGAAACAAATGAATGATGCACATTATC |

TABLE 13
Oligonucleotides for
detecting mutation M1043I

| SEQ ID NO: | SEQUENCE 5′-3′ |
|---|---|
| 218 | AGCCACCATGATGTGCATCATTC |
| 219 | AGCCACCATGATGTGCATCATTA |
| 220 | AGCCACCATGATGTGCATCATAA |
| 221 | AGCCACCATGATGTGCATCATGA |
| 222 | AGCCACCATGATGTGCATCAATA |
| 223 | AGCCACCATGATGTGCATCACTA |
| 224 | AGCCACCATCATGTGCATCAGTA |
| 225 | AGCCACCATGATGTGCATCCTTA |
| 226 | AGCCACCATGATGTGCATCGTTA |
| 227 | AGCCACCATCATSTGCATCTTTA |
| 228 | AGGCACCATGATGTGCATCATTA |
| 229 | AGGCACCATGATGTGCATCATTA* |
| 230 | AGGCACCATGATGTGCATCA*TTA |

An embodiment of the present invention is an oligonucleotide for detecting a mutation at one or more nucleotide positions between codons 1042 and 1050 in the PIK3CA gene being at least 90% identical to and having the 3′-terminal nucleotide of one or more of the sequences selected from the group consisting of SEQ ID NOs: 2, 18, 39, 208 and 219. The oligonucleotides might comprise 3 or fewer mismatches with one of said sequences, excluding the 3′-terminal nucleotide and/or at least one mismatch among the penultimate 5 nucleotides at the 3′-terminus. The oligonucleotides might further comprise at least one modified nucleotide among the terminal 5 nucleotides at the 3′-terminus. In some embodiment, the oligonucleotides suitable for detecting one or more of the mutations M1043I, H1047L, H1047R, H1047Y and/or H1049R.

Another embodiment of the present invention is an oligonucleotide for detecting mutation N345K in the PIK3CA gene being at least 90% identical to and having the 3′-terminal nucleotide of SEQ ID NO: 61. The oligonucleotides might comprise 3 or fewer mismatches with SEQ ID NO: 61, excluding the 3′-terminal nucleotide and/or at least one mismatch among the penultimate 5 nucleotides at the 3′-terminus. The oligonucleotides might further comprise at least one modified nucleotide among the terminal 5 nucleotides at the 3′-terminus.

Another embodiment of the present invention is an oligonucleotide for detecting a mutation at one or more nucleotide position(s) between codons 541 and 547 in the PIK3CA gene being at least 90% identical to and having the 3′-terminal nucleotide of one or more of the sequences selected from the group consisting of SEQ ID NOs: 84, 99, 126, 148, 168, 185 and 197. The oligonucleotides might comprise 3 or fewer mismatches with one of said sequences, excluding the 3′-terminal nucleotide and/or at least one mismatch among the penultimate 5 nucleotides at the 3′-terminus. The oligonucleotides might further comprise at least one modified nucleotide among the terminal 5 nucleotides at the 3′-terminus. The oligonucleotides are in particular suitable for detecting one or more of the mutations E542K, E545A, E545G, E545K, Q546K, Q546L and/or Q546E.

In another embodiment, the present invention is a diagnostic method of detecting mutations in the human PI3KCA (PIK3CA) gene selected from H1047L, H1047R, H1047Y, N345K, E542K, E545A, E545G, E545K, G1049R, M1043I, Q546E, Q546L and Q546K as well as a simultaneous query for 1, 2, 3, 4, 5, 6, 7, 8, 9, 10, 11, 12 or 13 of the mutations listed above using oligonucleotides selected from SEQ ID NOs: 2, 18, 39, 61, 84, 100, 127, 148, 170, 185, 197, 208, 219 or variations at least 90% identical to and having the 3′-terminal nucleotide of said oligonucleotides. In variations of this embodiment, the method comprises using one or more oligonucleotides selected from SEQ ID NOs: 8, 21, 46, 78, 93, 113, 141, 166, 170, 194, 199, 217 and 228. The method comprises contacting a test sample containing nucleic acids with one or more of the oligonucleotides in the presence of the corresponding downstream primer and a detection probe. Advantageously, detection of closely positioned mutations can be performed in a single reaction. In some embodiments, a single reaction contains two or more allele-specific oligonucleotides, e.g., SEQ ID NOs: 8, 21 and 46 can be combined in one reaction mixture together with a single downstream primer and a single detection probe. Similarly, a single reaction may contain two or more of SEQ ID NOs: 93, 113, 141, 166, 170 and 199 can be combined in one reaction mixture together with a single downstream primer and a single detection probe The method comprises contacting a test sample containing nucleic acids with one or more of the oligonucleotides in the presence of the corresponding downstream primer (i.e. a primer capable of hybridizing to the opposite strand of the target nucleic acid so as to enable exponential amplification), nucleoside triphosphates and a nucleic acid polymerase, such that the one or more allele-specific primers is efficiently extended only when an PI3KCA mutation is present in the sample; and detecting the presence or absence of an PI3KCA mutation by directly or indirectly detecting the presence or absence of the primer extension.

In a particular embodiment the presence of the primer extension is detected with a probe. The probe may be labeled with a radioactive, or a chromophore (fluorophore) label, e.g. a label incorporating FAM, JA270, CY5 family dyes, or HEX dyes. As one example of detection using a fluorescently labeled probe, the mutation may be detected by real-time polymerase chain reaction (rt-PCR), where hybridization of the probe results in enzymatic digestion of the probe and detection of the resulting fluorescence (TaqMan™ probe method, Holland et al. (1991) P.N.A.S. USA 88:7276-7280). Alternatively, the presence of the extension product and the amplification product may be detected by gel electrophoresis followed by staining or by blotting and hybridization as described e.g., in Sambrook, J. and Russell, D. W. (2001) Molecular Cloning, 3^{rd} ed. CSHL Press, Chapters 5 and 9.

In another embodiment, the invention is a method of treating a patient having a tumor possibly harboring cells with a mutant PI3KCA gene. The method comprises contacting a sample from the patient with one or more oligonucleotides selected from SEQ ID NOs: 2, 18, 39, 61, 84, 100, 127, 148, 170, 185, 197, 208, 219 or variations at least 90% identical to and having the 3′-terminal nucleotide of said oligonucleotides, in the presence of a corresponding second primer or primers, conducting allele-specific amplification, and detecting the presence or absence of an PI3KCA mutation by detecting presence or absence of the primer extension, and if at least one mutation is found or not found, subjecting the patient the appropriate treatment regimen. In some embodiments, the treatment comprises administering an inhibitor of the protein encoded by PI3KCA gene (p110-alpha protein). In other embodiments, the treatment comprises administering an inhibitor of a protein upstream in the pathway, e.g. the EGFR protein, if PI3KCA mutations are not found and administering an alternative treatment if the mutations are found. In variations of this embodiment, the method comprises contacting a sample from the patient with one or more oligonucleotides selected from SEQ ID NOs: 8, 21, 46, 78, 93, 113, 141, 166, 170, 194, 199, 217 and 228.

In yet another embodiment, the invention is a kit containing reagents for detecting mutations in the PI3KCA gene, specifically the mutations selected from H1047L, H1047R, H1047Y, N345K, E542K, E545A, E545G, E545K, G1049R, M1043I, Q546E, Q546L and Q546K as well as a simultaneous query for 1, 2, 3, 4, 5, 6, 7, 8, 9, 10, 11, 12 or 13 of the mutations listed above. The reagents comprise one or more oligonucleotides selected from SEQ ID NOs: 2, 18, 39, 61, 84, 100, 127, 148, 170, 185, 197, 208, 219 or variations at least 90% identical to and having the 3′-terminal nucleotide of said oligonucleotides, one or more corresponding second primers, and optionally, one or more probes. In variations of this embodiment, the reagents comprise one or more oligonucleotides selected from SEQ ID NOs: 11, 32, 46, 78, 93, 113, 141, 166, 170, 194, 199, 217 and 228. The kit may further comprise reagents necessary for the performance of amplification and detection assay, such as nucleoside triphosphates, nucleic acid polymerase and buffers necessary for the function of the polymerase. In some embodiments, the probe is detectably labeled. In such embodiments, the kit may comprise reagents for labeling and detecting the label.

In yet another embodiment, the invention is a reaction mixture for detecting mutations in the PI3KCA gene, specifically the mutations selected from H1047L, H1047R, H1047Y, N345K, E542K, E545A, E545G, E545K, G1049R, M1043I, Q546E, Q546L and Q546K as well as a simultaneous query for 1, 2, 3, 4, 5, 6, 7, 8, 9, 10, 11, 12 or 13 of the mutations listed above. The mixture comprises one or more oligonucleotides selected from SEQ ID NOs: 2, 18, 39, 61, 84, 100, 127, 148, 170, 185, 197, 208, 219 or variations at least 90% identical to and having the 3′-terminal nucleotide of said oligonucleotides, one or more corresponding second primers, and optionally, one or more probes. In variations of this embodiment, the reaction mixture comprises one or more oligonucleotides selected from SEQ ID NOs: 8, 21, 46, 78, 93, 113, 141, 166, 170, 194, 199, 217 and 228. The reaction mixture may further comprise reagents such as nucleoside triphosphates, nucleic acid polymerase and buffers necessary for the function of the polymerase.

In yet another embodiment, the invention is a method of assessing cancer in patient by detecting in a patient's sample mutations in the PI3KCA gene, specifically the mutations selected from H1047L, H1047R, H1047Y, N345K, E542K, E545A, E545G, E545K, G1049R, M1043I, Q546E, Q546L and Q546K as well as a simultaneous query for 1, 2, 3, 4, 5, 6, 7, 8, 9, 10, 11, 12 or 13 of the mutations listed above, for each mutation using an oligonucleotide selected from SEQ ID NOs: 2, 18, 39, 61, 84, 100, 127, 148, 170, 185, 197, 208, 219 or variations at least 90% identical to and having the 3′-terminal nucleotide of said oligonucleotides. In variations of this embodiment, the oligonucleotides are selected from SEQ ID NOs: 8, 21, 46, 78, 93, 113, 141, 166, 170, 194, 199, 217 and 228.

### EXAMPLES

### Exemplary Reaction Conditions

In all examples below, the following reaction conditions were used. Each reaction included the 10⁴ copies or mutant or wild-type DNA template, 0.1 μM each of selective and common primer, detection probe, uracil-N-glycosylase, DNA polymerase and a suitable DNA polymerase buffer. The reactions were subjected to the following thermal cycling profile on the LIGHTCYCLER® 480 instrument (Roche Molecular Diagnostics, Indianapolis, Ind.): 50° C. for 5 minutes, followed by 2 cycles of 95° C. (10 seconds) to 62° C. (30 seconds), and 65 cycles of 93° C. (10 seconds) to 62° C. (30 seconds). Fluorescence data was collected at the start of each 62° C. step. Cₜ values from each reaction were used to calculate ΔCₜ. Average Ct and standard deviation are shown for each example.

### Example 1

### Performance of Primers for Detecting Mutation H1047L in the Human PI3KCA Gene

| SEQ ID NO: | Wt Cₜ | St dev | Mut Cₜ | St dev | Δ Cₜ |
|---|---|---|---|---|---|
| 1 (WT match) | 18.4 | 0.1 | 24.1 | 0.3 | −5.7 |
| 2 | 31.8 | 0.2 | 18.8 | 0.1 | 13.0 |
| 3 | 43.2 | 3.0 | 18.9 | 0.1 | 24.3 |
| 4 | 35.6 | 0.5 | 20.8 | 0.0 | 14.8 |
| 5 | 45.1 | 5.3 | 19.7 | 0.0 | 25.4 |
| 6 | 61.9 | 5.2 | 21.0 | 0.0 | 40.9 |
| 7 | 38.8 | 1.4 | 19.7 | 0.1 | 19.1 |
| 8 | 55.3 | 6.0 | 19.8 | 0.1 | 35.5 |
| 9 | 49.2 | 5.3 | 18.8 | 0.1 | 30.3 |
| 10 | 44.8 | 5.2 | 19.0 | 0.0 | 25.8 |
| 11 | 47.8 | 1.4 | 18.9 | 0.0 | 28.9 |
| 12 | 31.8 | 0.2 | 19.7 | 0.1 | 12.1 |
| 13 | 39.6 | 1.5 | 19.9 | 0.0 | 19.7 |
| 14 | 18.4 | 0.1 | 24.1 | 0.3 | −5.7 |
| 15 | 31.8 | 0.2 | 18.8 | 0.1 | 13.0 |
| 16 | 43.2 | 3.0 | 18.9 | 0.1 | 24.3 |

### Example 2

### Performance of Primers for Detecting Mutation H1047R in the Human PI3KCA Gene

| SEQ ID NO: | Wt Cₜ | St dev | Mut Cₜ | St dev | Δ Cₜ |
|---|---|---|---|---|---|
| 17 (WT match) | 18.2 | 0.2 | 20.4 | 0.0 | −2.2 |
| 18 | 32.2 | 0.3 | 18.9 | 0.0 | 13.3 |
| 19 | 31.7 | 0.3 | 19.5 | 0.0 | 12.2 |
| 20 | 33.9 | 0.4 | 19.5 | 0.0 | 14.4 |
| 21 | 35.5 | 1.0 | 20.1 | 0.3 | 15.4 |
| 22 | 32.7 | 0.7 | 19.4 | 0.1 | 13.3 |
| 23 | 33.7 | 0.6 | 19.5 | 0.0 | 14.2 |
| 24 | 31.9 | 0.4 | 18.9 | 0.0 | 12.9 |
| 25 | 29.8 | 1.0 | 18.9 | 0.1 | 10.9 |
| 26 | 35.4 | 0.7 | 22.3 | 0.1 | 13.2 |
| 27 | 37.1 | 0.6 | 22.8 | 0.0 | 14.3 |
| 28 | 37.0 | 0.7 | 22.7 | 0.0 | 14.3 |
| 29 | 41.7 | 5.1 | 22.3 | 0.1 | 19.4 |
| 30 | 40.2 | 2.4 | 24.7 | 0.2 | 15.5 |
| 31 | 43.4 | 3.2 | 25.1 | 0.5 | 18.3 |
| 32 | 65.0 | 0.0 | 33.5 | 0.0 | 31.5 |
| 33 | 38.8 | 0.9 | 23.1 | 0.3 | 15.8 |
| 34 | 38.4 | 0.7 | 23.1 | 0.3 | 15.2 |
| 35 | 43.0 | 1.8 | 24.6 | 0.5 | 18.5 |
| 36 | 40.4 | 0.0 | 22.7 | 0.2 | 17.8 |
| 37 | 38.3 | 0.9 | 23.2 | 0.0 | 15.1 |

### Example 3

### Performance of Primers for Detecting Mutation H1047Y in the Human PI3KCA Gene

| SEQ ID NO: | Wt Cₜ | St dev | Mut Cₜ | St dev | Δ Cₜ |
|---|---|---|---|---|---|
| 38 (WT match) | 18.0 | 0.2 | 19.3 | 0.0 | −1.3 |
| 39 | 47.5 | 2.9 | 22.6 | 0.0 | 24.9 |
| 40 | 32.8 | 0.3 | 19.3 | 0.0 | 13.5 |
| 41 | 37.3 | 2.4 | 24.6 | 0.1 | 12.7 |
| 42 | 33.2 | 0.4 | 18.9 | 0.0 | 14.3 |
| 43 | 34.4 | 0.8 | 19.4 | 0.0 | 15.0 |
| 44 | 35.0 | 0.9 | 19.1 | 0.2 | 15.9 |
| 45 | 36.1 | 2.4 | 20.2 | 0.0 | 15.9 |
| 46 | 36.0 | 0.4 | 19.5 | 0.2 | 16.5 |
| 47 | 35.8 | 0.8 | 19.5 | 0.1 | 16.3 |
| 48 | 30.0 | 0.2 | 19.6 | 0.1 | 10.4 |
| 49 | 34.4 | 0.5 | 19.7 | 0.0 | 14.7 |
| 50 | 33.3 | 0.3 | 20.4 | 0.0 | 12.9 |
| 51 | 45.8 | 7.9 | 23.3 | 0.0 | 22.5 |
| 52 | 45.1 | 7.6 | 21.8 | 0.1 | 23.4 |
| 53 | 52.9 | 6.5 | 24.1 | 0.1 | 28.8 |
| 54 | 60.8 | 10.4 | 27.2 | 0.0 | 33.5 |
| 55 | 34.7 | 1.2 | 19.3 | 0.0 | 15.4 |
| 56 | 33.1 | 1.3 | 19.8 | 0.0 | 13.3 |
| 57 | 38.2 | 1.3 | 22.9 | 0.7 | 15.3 |
| 58 | 65.0 | 0.0 | 42.5 | 0.3 | 22.5 |
| 59 | 61.6 | 6.1 | 24.6 | 0.1 | 37.0 |

### Example 4

### Performance of Primers for Detecting Mutation N345K in the Human PI3KCA Gene

| SEQ ID NO: | Wt Cₜ | St dev | Mut Cₜ | St dev | Δ Cₜ |
|---|---|---|---|---|---|
| 60 (WT match) | 21.6 | 0.1 | 27.4 | 0.0 | −5.8 |
| 61 | 34.1 | 0.1 | 23.8 | 0.0 | 10.3 |
| 62 | 44.2 | 1.0 | 24.0 | 0.1 | 20.2 |
| 63 | 43.5 | 1.6 | 25.4 | 0.1 | 18.0 |
| 64 | 44.0 | 1.3 | 24.4 | 0.1 | 19.6 |
| 65 | 33.7 | 0.3 | 23.9 | 0.1 | 9.8 |
| 66 | 25.5 | 0.3 | 23.2 | 0.1 | 2.3 |
| 67 | 33.9 | 0.4 | 23.8 | 0.0 | 10.1 |
| 68 | 44.7 | 1.4 | 25.9 | 0.1 | 18.8 |
| 69 | 39.7 | 0.3 | 25.7 | 0.0 | 14.0 |
| 70 | 47.9 | 1.1 | 27.0 | 0.6 | 20.9 |
| 71 | 35.2 | 0.5 | 23.6 | 0.0 | 11.5 |
| 72 | 55.7 | 9.4 | 24.6 | 0.0 | 31.1 |
| 73 | 60.7 | 8.2 | 27.5 | 0.2 | 33.2 |
| 74 | 32.6 | 0.4 | 23.4 | 0.3 | 9.2 |
| 75 | 65.0 | 0.0 | 29.6 | 0.0 | 35.4 |
| 76 | 29.6 | 0.2 | 26.6 | 0.1 | 3.0 |
| 77 | 58.8 | 4.0 | 25.4 | 0.1 | 33.4 |
| 78 | 65.0 | 0.0 | 25.4 | 2.1 | 39.6 |
| 79 | 45.4 | 4.2 | 27.5 | 0.1 | 17.8 |
| 80 | 62.4 | 6.3 | 29.7 | 0.3 | 32.8 |
| 81 | 51.4 | 5.2 | 26.0 | 0.1 | 25.4 |
| 82 | 65.0 | 0.0 | 40.8 | 0.0 | 24.2 |

### Example 5

### Performance of Primers for Detecting Mutation E542K in the Human PI3KCA Gene

| SEQ ID NO: | Wt Cₜ | St dev | Mut Cₜ | St dev | Δ Cₜ |
|---|---|---|---|---|---|
| 83 (WT match) | 23.9 | 0.5 | 30.8 | 0.2 | −6.9 |
| 84 | 37.4 | 0.2 | 24.4 | 0.0 | 13.0 |
| 85 | 64.6 | 1.0 | 30.8 | 0.3 | 33.8 |
| 86 | 55.7 | 5.0 | 27.6 | 0.0 | 28.1 |
| 87 | 38.4 | 0.5 | 37.3 | 0.5 | 1.2 |
| 88 | 48.0 | 1.4 | 45.3 | 0.5 | 2.7 |
| 89 | 60.8 | 4.8 | 29.1 | 0.8 | 31.7 |
| 90 | 58.5 | 6.2 | 28.3 | 0.1 | 30.2 |
| 91 | 49.6 | 3.3 | 24.4 | 0.1 | 25.2 |
| 92 | 46.7 | 1.8 | 24.4 | 0.1 | 22.2 |
| 93 | 52.9 | 6.1 | 25.0 | 0.5 | 27.9 |
| 94 | 39.5 | 0.8 | 24.2 | 0.3 | 15.3 |
| 95 | 65.0 | 0.0 | 32.8 | 0.2 | 32.2 |
| 96 | 47.0 | 1.2 | 23.6 | 0.1 | 23.4 |
| 97 | 50.8 | 3.2 | 46.5 | 1.9 | 4.3 |

### Example 6

### Performance of Primers for Detecting Mutation E545A in the Human PI3KCA Gene

| SEQ ID NO: | Wt Cₜ | St dev | Mut Cₜ | St dev | Δ Cₜ |
|---|---|---|---|---|---|
| 98 (WT match) | 23.9 | 0.3 | 35.1 | 0.2 | −11.2 |
| 99 | 24.4 | 0.2 | 24.1 | 0.2 | 0.3 |
| 100 | 48.4 | 3.8 | 25.6 | 0.0 | 22.9 |
| 101 | 47.6 | 3.5 | 25.6 | 0.3 | 22.0 |
| 102 | 42.6 | 1.2 | 24.9 | 0.0 | 17.7 |
| 103 | 35.5 | 1.0 | 24.5 | 0.2 | 11.0 |
| 104 | 39.3 | 1.5 | 24.6 | 0.0 | 14.7 |
| 105 | 35.8 | 1.0 | 24.7 | 0.1 | 11.1 |
| 106 | 32.4 | 0.5 | 24.4 | 0.1 | 8.0 |
| 107 | 36.2 | 0.8 | 24.7 | 0.0 | 11.5 |
| 108 | 40.5 | 0.8 | 24.8 | 0.2 | 15.7 |
| 109 | 24.9 | 0.6 | 23.7 | 0.2 | 1.3 |
| 110 | 35.2 | 0.8 | 25.5 | 1.7 | 9.7 |
| 111 | 31.0 | 0.7 | 22.9 | 0.0 | 8.2 |
| 112 | 41.6 | 1.7 | 23.5 | 0.1 | 18.1 |
| 113 | 45.1 | 1.1 | 23.8 | 0.0 | 21.3 |
| 114 | 65.0 | 0.0 | 35.6 | 0.0 | 29.4 |
| 115 | 65.0 | 0.0 | 65.0 | 0.0 | 0.0 |
| 116 | 60.7 | 1.2 | 34.3 | 0.1 | 26.4 |
| 117 | 65.0 | 0.0 | 48.7 | 0.3 | 16.3 |
| 118 | 65.5 | 1.5 | 35.3 | 0.3 | 30.2 |
| 119 | 64.9 | 0.3 | 33.6 | 0.4 | 31.3 |
| 120 | 65.0 | 0.0 | 34.3 | 0.4 | 30.7 |
| 121 | 65.0 | 0.0 | 58.2 | 9.6 | 6.8 |
| 122 | 56.6 | 4.6 | 33.4 | 0.1 | 23.2 |
| 123 | 65.0 | 0.0 | 39.7 | 0.0 | 25.3 |
| 124 | 60.1 | 1.8 | 34.1 | 0.5 | 26.0 |

### Example 7

### Performance of Primers for Detecting Mutation E545G in the Human PI3KCA Gene

| SEQ ID NO: | Wt Cₜ | St dev | Mut Cₜ | St dev | Δ Cₜ |
|---|---|---|---|---|---|
| 125 (WT match) | 23.7 | 0.5 | 25.1 | 0.2 | −1.4 |
| 126 | 24.3 | 0.3 | 24.2 | 0.1 | 0.0 |
| 127 | 43.4 | 0.5 | 24.9 | 0.0 | 18.4 |
| 128 | 28.1 | 0.9 | 25.1 | 0.2 | 3.0 |
| 129 | 38.3 | 1.7 | 24.9 | 0.1 | 13.4 |
| 130 | 37.7 | 0.3 | 24.5 | 0.0 | 13.2 |
| 131 | 35.1 | 0.6 | 24.3 | 0.2 | 10.9 |
| 132 | 37.5 | 1.2 | 24.5 | 0.2 | 13.0 |
| 133 | 29.8 | 0.5 | 24.3 | 0.1 | 5.5 |
| 134 | 35.9 | 0.3 | 24.6 | 0.1 | 11.3 |
| 135 | 33.6 | 0.6 | 24.2 | 0.3 | 9.5 |
| 136 | 24.5 | 0.4 | 23.7 | 0.1 | 0.8 |
| 137 | 31.0 | 0.4 | 25.8 | 1.5 | 5.2 |
| 138 | 54.3 | 7.8 | 25.4 | 0.3 | 28.9 |
| 139 | 65.0 | 0.0 | 39.0 | 0.0 | 26.0 |
| 140 | 56.3 | 4.7 | 30.0 | 0.2 | 26.3 |
| 141 | 43.9 | 0.9 | 23.7 | 0.3 | 20.3 |
| 142 | 43.8 | 1.1 | 24.1 | 0.3 | 19.7 |
| 143 | 65.0 | 0.0 | 39.2 | 0.4 | 25.8 |
| 144 | 30.2 | 1.0 | 26.3 | 0.0 | 4.0 |
| 145 | 65.0 | 0.0 | 65.0 | 0.0 | 0.0 |
| 146 | 37.9 | 0.7 | 23.8 | 0.2 | 14.1 |

### Example 8

### Performance of Primers for Detecting Mutation E545K in the Human PI3KCA Gene

| SEQ ID NO: | Wt Cₜ | St dev | Mut Cₜ | St dev | Δ Cₜ |
|---|---|---|---|---|---|
| 147 (WT match) | 23.5 | 0.3 | 25.4 | 0.2 | −1.9 |
| 148 | 29.1 | 0.5 | 23.9 | 0.0 | 5.2 |
| 149 | 45.7 | 2.7 | 26.4 | 0.0 | 19.3 |
| 150 | 42.1 | 1.1 | 24.9 | 0.1 | 17.2 |
| 151 | 26.5 | 0.5 | 30.9 | 0.1 | −4.3 |
| 152 | 40.7 | 1.7 | 24.4 | 0.1 | 16.3 |
| 153 | 47.3 | 2.3 | 26.7 | 0.0 | 20.6 |
| 154 | 44.7 | 2.0 | 25.7 | 0.2 | 19.0 |
| 155 | 37.9 | 0.9 | 24.0 | 0.3 | 13.9 |
| 156 | 35.8 | 0.5 | 24.1 | 0.2 | 11.7 |
| 157 | 41.6 | 1.1 | 24.1 | 0.2 | 17.5 |
| 158 | 31.2 | 0.7 | 24.9 | 0.0 | 6.3 |
| 159 | 56.3 | 1.6 | 34.5 | 0.4 | 21.9 |
| 160 | 28.2 | 0.6 | 22.9 | 0.4 | 5.3 |
| 161 | 45.4 | 1.5 | 25.6 | 0.3 | 19.8 |
| 162 | 51.2 | 5.7 | 29.8 | 0.2 | 21.4 |
| 163 | 58.7 | 7.0 | 32.0 | 0.0 | 26.8 |
| 164 | 48.7 | 2.7 | 29.5 | 0.1 | 19.2 |
| 165 | 49.5 | 7.1 | 30.5 | 0.4 | 19.0 |
| 166 | 42.0 | 2.0 | 24.5 | 0.0 | 17.5 |

### Example 9

### Performance of Primers for Detecting Mutation Q546K in the Human PI3KCA Gene

| SEQ ID NO: | Wt Cₜ | St dev | Mut Cₜ | St dev | Δ Cₜ |
|---|---|---|---|---|---|
| 167 (WT match) | 23.3 | 0.5 | 25.8 | 0.1 | −2.5 |
| 168 | 34.9 | 0.4 | 41.8 | 0.6 | −6.9 |
| 169 | 26.2 | 0.6 | 28.4 | 0.0 | −2.2 |
| 170 | 55.2 | 2.8 | 25.9 | 0.0 | 29.2 |
| 171 | 44.6 | 1.8 | 26.7 | 0.1 | 17.9 |
| 172 | 42.1 | 1.0 | 24.6 | 0.3 | 17.4 |
| 173 | 37.2 | 1.1 | 25.3 | 0.1 | 11.9 |
| 174 | 35.3 | 1.3 | 24.5 | 0.0 | 10.7 |
| 175 | 65.0 | 0.0 | 65.0 | 0.0 | 0.0 |
| 176 | 44.7 | 0.9 | 26.6 | 0.2 | 18.1 |
| 177 | 41.5 | 0.4 | 26.0 | 0.2 | 15.6 |
| 178 | 37.4 | 0.9 | 44.5 | 0.3 | −7.0 |
| 179 | 65.0 | 0.0 | 65.0 | 0.0 | 0.0 |
| 180 | 65.0 | 0.0 | 65.0 | 0.0 | 0.0 |
| 181 | 48.9 | 2.9 | 25.2 | 0.1 | 23.7 |
| 182 | 33.4 | 0.3 | 24.4 | 0.0 | 9.0 |
| 183 | 40.1 | 1.1 | 25.8 | 0.1 | 14.3 |

### Example 10

### Performance of Primers for Detecting Mutation Q546E in the Human PI3KCA Gene

| SEQ ID NO: | Wt Cₜ | St dev | Mut Cₜ | St dev | Δ Cₜ |
|---|---|---|---|---|---|
| 184 (WT match) | 21.5 | 0.4 | 31.9 | 0.5 | −10.4 |
| 185 | 30.8 | 0.5 | 22.6 | 0.0 | 8.2 |
| 186 | 36.7 | 1.2 | 24.6 | 0.1 | 12.1 |
| 187 | 39.0 | 0.6 | 27.8 | 0.0 | 11.1 |
| 188 | 56.8 | 5.8 | 27.6 | 0.4 | 29.2 |
| 189 | 38.3 | 0.7 | 23.5 | 0.1 | 14.8 |
| 190 | 35.0 | 0.4 | 23.9 | 0.1 | 11.2 |
| 191 | 43.7 | 0.8 | 23.8 | 0.1 | 19.9 |
| 192 | 50.5 | 3.8 | 25.3 | 0.4 | 25.3 |
| 193 | 38.3 | 0.8 | 26.4 | 0.1 | 11.8 |
| 194 | 52.7 | 3.1 | 24.7 | 0.1 | 28.1 |

### Example 11

### Performance of Primers for Detecting Mutation Q546L in the Human PI3KCA Gene

| SEQ ID NO: | Wt Cₜ | St dev | Mut Cₜ | St dev | Δ Cₜ |
|---|---|---|---|---|---|
| 195 (WI match) | 22.2 | 0.4 | 33.9 | 0.1 | −11.7 |
| 197 | 31.2 | 0.7 | 21.9 | 0.1 | 9.3 |
| 198 | 63.0 | 2.5 | 29.5 | 0.1 | 33.5 |
| 199 | 56.5 | 3.0 | 23.2 | 0.3 | 33.3 |
| 200 | 47.1 | 2.3 | 22.8 | 0.0 | 24.2 |
| 201 | 56.4 | 5.6 | 23.1 | 0.2 | 33.3 |
| 202 | 51.9 | 4.6 | 24.3 | 0.2 | 27.6 |
| 203 | 55.9 | 2.6 | 23.5 | 0.2 | 32.4 |
| 204 | 42.4 | 0.6 | 22.6 | 0.1 | 19.8 |
| 205 | 39.7 | 0.9 | 22.4 | 0.4 | 17.3 |
| 206 | 44.8 | 1.7 | 22.4 | 0.2 | 22.4 |

### Example 12

### Performance of Primers for Detecting Mutation G1049R in the Human PI3KCA Gene

| SEQ ID NO: | Wt Cₜ | St dev | Mut Cₜ | St dev | Δ Cₜ |
|---|---|---|---|---|---|
| 207 (WT match) | 18.8 | 0.1 | 30.3 | 0.1 | −11.5 |
| 208 | 32.2 | 0.4 | 20.4 | 0.0 | 11.8 |
| 209 | 36.1 | 0.5 | 22.3 | 0.1 | 13.8 |
| 210 | 41.6 | 2.1 | 20.7 | 0.1 | 20.9 |
| 211 | 43.4 | 1.6 | 26.4 | 0.0 | 16.9 |
| 212 | 49.9 | 8.2 | 20.1 | 0.3 | 29.8 |
| 213 | 48.3 | 5.6 | 20.4 | 0.1 | 28.0 |
| 214 | 41.3 | 2.2 | 20.8 | 0.0 | 20.5 |
| 215 | 43.5 | 4.9 | 20.8 | 0.1 | 22.8 |
| 216 | 48.3 | 2.9 | 20.7 | 0.1 | 27.7 |
| 217 | 53.0 | 5.1 | 20.5 | 0.1 | 32.5 |

### Example 13

### Performance of Primers for Detecting Mutation M1043I in the Human PI3KCA Gene

| SEQ ID NO: | Wt Cₜ | St dev | Mut Cₜ | St dev | Δ Cₜ |
|---|---|---|---|---|---|
| 218 | 17.8 | 0.1 | 30.8 | 0.0 | −13.0 |
| 219 | 46.7 | 4.2 | 19.5 | 0.1 | 27.1 |
| 220 | 53.8 | 6.5 | 26.7 | 0.0 | 27.1 |
| 221 | 51.0 | 9.1 | 32.1 | 0.3 | 19.0 |
| 222 | 38.7 | 2.5 | 22.0 | 0.0 | 16.7 |
| 223 | 52.1 | 5.0 | 21.7 | 0.0 | 30.4 |
| 224 | 50.6 | 5.2 | 25.6 | 0.0 | 25.0 |
| 225 | 51.5 | 9.1 | 20.5 | 0.0 | 31.0 |
| 226 | 52.0 | 2.5 | 19.7 | 0.0 | 32.3 |
| 227 | 50.0 | 5.0 | 20.5 | 0.0 | 29.5 |
| 228 | 61.5 | 5.8 | 20.1 | 0.0 | 41.3 |
| 229 | 65.0 | 0.0 | 35.5 | 0.0 | 29.5 |
| 230 | 65.0 | 0.0 | 23.2 | 0.1 | 41.8 |

While the invention has been described in detail with reference to specific examples, it will be apparent to one skilled in the art that various modifications can be made within the scope of this invention. Thus the scope of the invention should not be limited by the examples described herein, but by the claims presented below.

What is claimed is:

1. A set of oligonucleotides for detecting one or more mutations in the human PIK3CA gene comprising:
(i) a first labeled oligonucleotide detection probe;
(ii) a first oligonucleotide primer having at least 90% identity to, and having the same 3′-terminal nucleotide as SEQ ID NO:18 and comprising at least one mismatch with the polynucleotide sequence encoding wild type or H1047 mutant human PIK3CA gene among the penultimate 5 nucleotides at the 3′-terminus of the first oligonucleotide primer;
(iii) a second labeled oligonucleotide detection probe; and
(iv) a second oligonucleotide primer having at least 90% identity to, and having the same 3′-terminal nucleotide as SEQ ID NO:61, and comprising at least one mismatch with the polynucleotide sequence encoding wild type or N345K mutant human PIK3CA gene among the penultimate 5 nucleotides at the 3′-terminus of the second oligonucleotide primer.

2. The set of oligonucleotides of claim 1, wherein at least one oligonucleotide comprises at least one nucleotide with a modified base selected from N4-methyl-deoxycytosine, N4-ethyl-deoxycytosine, N4-tert-butyl-benzyl-deoxycytosine, and N6-tert-butyl-benzyl-deoxyadenine.

3. The set of oligonucleotides of claim 1, wherein the oligonucleotide primer of (ii) has the sequence of SEQ ID NO:21.

4. The set of oligonucleotides of claim 1, wherein the oligonucleotide primer of (iv) has the sequence of SEQ ID NO:78.

5. The set of oligonucleotides of claim 1, comprising oligonucleotide primers having the sequences of SEQ ID NO:21 and SEQ ID NO:78.

6. The set of oligonucleotides of claim 1, wherein the first or second labeled oligonucleotide probe is fluorescently labeled.

7. The set of oligonucleotides of claim 1, further comprising at least one additional oligonucleotide having a sequence selected from the group consisting of SEQ ID NOs:8, 46, 93, 113, 141, 166, 170, 194, 199, 217 and 228.

8. A reaction mixture for detecting one or more mutations in the human PIK3CA gene comprising:
(i) a first labeled oligonucleotide detection probe;
(ii) a first oligonucleotide primer having at least 90% identity to, and having the same 3′-terminal nucleotide as SEQ ID NO:18 and comprising at least one mismatch with the polynucleotide sequence encoding wild type or H1047 mutant human PIK3CA gene among the penultimate 5 nucleotides at the 3′-terminus of the first oligonucleotide primer;
(iii) a second labeled oligonucleotide detection probe;
(iv) a second oligonucleotide primer having at least 90% identity to, and having the same 3′-terminal nucleotide as SEQ ID NO:61, and comprising at least one mismatch with the polynucleotide sequence encoding wild type or N345K mutant human PIK3CA gene among the penultimate 5 nucleotides at the 3′-terminus of the second oligonucleotide primer; and
(v) DNA polymerase.

9. The reaction mixture of claim 8, further comprising uracil-N-glycosylase.

10. The reaction mixture of claim 8, wherein at least one oligonucleotide comprises at least one nucleotide with a modified base selected from N4-methyl-deoxycytosine, N4-ethyl-deoxycytosine, N4-tert-butyl-benzyl-deoxycytosine, and N6-tert-butyl-benzyl-deoxyadenine.

11. The reaction mixture of claim 8, wherein the oligonucleotide primer of (ii) has the sequence of SEQ ID NO:21.

12. The reaction mixture of claim 8, wherein the oligonucleotide primer of (iv) has the sequence of SEQ ID NO:78.

13. The reaction mixture of claim 8, comprising oligonucleotide primers having the sequences of SEQ ID NO:21 and SEQ ID NO:78.

14. The reaction mixture of claim 8, wherein the labeled oligonucleotide probe is fluorescently labeled.

15. The reaction mixture of claim 8, further comprising at least one additional oligonucleotide having a sequence selected from the group consisting of SEQ ID NOs:8, 46, 93, 113, 141, 166, 170, 194, 199, 217 and 228.


SEQUENCE LISTING (230 sequences)

SEQ ID NO: 1 - DNA, 23 bp, Artificial Sequence
  Description of Artificial Sequence: Synthetic primer
ttttgttgtccagccaccatgat

SEQ ID NO: 2 - DNA, 23 bp, Artificial Sequence
  Description of Artificial Sequence: Synthetic primer
ttttgttgtccagccaccatgaa

SEQ ID NO: 3 - DNA, 23 bp, Artificial Sequence
  Description of Artificial Sequence: Synthetic primer
ttttgttgtccagccaccatgca

SEQ ID NO: 4 - DNA, 23 bp, Artificial Sequence
  Description of Artificial Sequence: Synthetic primer
ttttgttgtccagccaccatgga

SEQ ID NO: 5 - DNA, 23 bp, Artificial Sequence
  Description of Artificial Sequence: Synthetic primer
ttttgttgtccagccaccatgta

SEQ ID NO: 6 - DNA, 23 bp, Artificial Sequence
  Description of Artificial Sequence: Synthetic primer
ttttgttgtccagccaccatcaa

SEQ ID NO: 7 - DNA, 23 bp, Artificial Sequence
  Description of Artificial Sequence: Synthetic primer
ttttgttgtccagccaccattaa

SEQ ID NO: 8 - DNA, 23 bp, Artificial Sequence
  Description of Artificial Sequence: Synthetic primer
ttttgttgtccagccaccataaa

SEQ ID NO: 9 - DNA, 23 bp, Artificial Sequence
  Description of Artificial Sequence: Synthetic primer
ttttgttgtccagccaccaagaa

SEQ ID NO: 10 - DNA, 23 bp, Artificial Sequence
  Description of Artificial Sequence: Synthetic primer
ttttgttgtccagccaccacgaa

SEQ ID NO: 11 - DNA, 23 bp, Artificial Sequence
  Description of Artificial Sequence: Synthetic primer
ttttgttgtccagccaccaggaa

SEQ ID NO: 12 - DNA, 25 bp, Artificial Sequence
  Description of Artificial Sequence: Synthetic primer
gtttttgttgtccagccaccatgaa

SEQ ID NO: 13 - DNA, 25 bp, Artificial Sequence
  Description of Artificial Sequence: Synthetic primer
  modified_base (25)..(25): N6-tert-butyl-benzyl-deoxyadenine
gtttttgttgtccagccaccatgaa

SEQ ID NO: 14 - DNA, 25 bp, Artificial Sequence
  Description of Artificial Sequence: Synthetic primer
  modified_base (24)..(24): N6-tert-butyl-benzyl-deoxyadenine
gtttttgttgtccagccaccatgaa

SEQ ID NO: 15 - DNA, 27 bp, Artificial Sequence
  Description of Artificial Sequence: Synthetic primer
  modified_base (2)..(2): N4-methyl-deoxycytosine
  modified_base (14)..(14): N4-methyl-deoxycytosine
  modified_base (18)..(18): N4-methyl-deoxycytosine
  modified_base (22)..(22): N4-methyl-deoxycytosine
  modified_base (26)..(26): N6-tert-butyl-benzyl-deoxyadenine
ccgtttttgttgtccagccaccatgaa

SEQ ID NO: 16 - DNA, 30 bp, Artificial Sequence
  Description of Artificial Sequence: Synthetic primer
  modified_base (5)..(5): N4-methyl-deoxycytosine
  modified_base (17)..(17): N4-methyl-deoxycytosine
  modified_base (21)..(21): N4-methyl-deoxycytosine
  modified_base (25)..(25): N4-methyl-deoxycytosine
  modified_base (30)..(30): N6-tert-butyl-benzyl-deoxyadenine
aatccattgttgttgtccagccaccatgaa

SEQ ID NO: 17 - DNA, 22 bp, Artificial Sequence
  Description of Artificial Sequence: Synthetic primer
tttgttgtccagccaccatgat

SEQ ID NO: 18 - DNA, 22 bp, Artificial Sequence
  Description of Artificial Sequence: Synthetic primer
tttgttgtccagccaccatgcc

SEQ ID NO: 19 - DNA, 22 bp, Artificial Sequence
  Description of Artificial Sequence: Synthetic primer
tttgttgtccagccaccatggc

SEQ ID NO: 20 - DNA, 22 bp, Artificial Sequence
  Description of Artificial Sequence: Synthetic primer
tttgttgtccagccaccatgtc

SEQ ID NO: 21 - DNA, 22 bp, Artificial Sequence
  Description of Artificial Sequence: Synthetic primer
tttgttgtccagccaccatcac

SEQ ID NO: 22 - DNA, 22 bp, Artificial Sequence
  Description of Artificial Sequence: Synthetic primer
tttgttgtccagccaccattac

SEQ ID NO: 23 - DNA, 22 bp, Artificial Sequence
  Description of Artificial Sequence: Synthetic primer
tttgttgtccagccaccataac

SEQ ID NO: 24 - DNA, 22 bp, Artificial Sequence
  Description of Artificial Sequence: Synthetic primer
tttgttgtccagccaccaagac

SEQ ID NO: 25 - DNA, 22 bp, Artificial Sequence
  Description of Artificial Sequence: Synthetic primer
tttgttgtccagccaccacgac

SEQ ID NO: 26 - DNA, 22 bp, Artificial Sequence
  Description of Artificial Sequence: Synthetic primer
tttgttgtccagccaccaggac

SEQ ID NO: 27 - DNA, 22 bp, Artificial Sequence
  Description of Artificial Sequence: Synthetic primer
tttgttgtccagccaccatgat

SEQ ID NO: 28 - DNA, 22 bp, Artificial Sequence
  Description of Artificial Sequence: Synthetic primer
tttgttgtccagccaccatgcc

SEQ ID NO: 29 - DNA, 27 bp, Artificial Sequence
  Description of Artificial Sequence: Synthetic primer
tttcatgaaacaaatgaatgatgcagg

SEQ ID NO: 30 - DNA, 27 bp, Artificial Sequence
  Description of Artificial Sequence: Synthetic primer
tttcatgaaacaaatgaatgatgcatg

SEQ ID NO: 31 - DNA, 27 bp, Artificial Sequence
  Description of Artificial Sequence: Synthetic primer
tttcatgaaacaaatgaatgatgcaag

SEQ ID NO: 32 - DNA, 27 bp, Artificial Sequence
  Description of Artificial Sequence: Synthetic primer
tttcatgaaacaaatgaatgatgcccg

SEQ ID NO: 33 - DNA, 27 bp, Artificial Sequence
  Description of Artificial Sequence: Synthetic primer
tttcatgaaacaaatgaatgatgcgcg

SEQ ID NO: 34 - DNA, 27 bp, Artificial Sequence
  Description of Artificial Sequence: Synthetic primer
tttcatgaaacaaatgaatgatgctcg

SEQ ID NO: 35 - DNA, 27 bp, Artificial Sequence
  Description of Artificial Sequence: Synthetic primer
tttcatgaaacaaatgaatgatggacg

SEQ ID NO: 36 - DNA, 27 bp, Artificial Sequence
  Description of Artificial Sequence: Synthetic primer
tttcatgaaacaaatgaatgatgtacg

SEQ ID NO: 37 - DNA, 27 bp, Artificial Sequence
  Description of Artificial Sequence: Synthetic primer
tttcatgaaacaaatgaatgatgaacg

SEQ ID NO: 38 - DNA, 23 bp, Artificial Sequence
  Description of Artificial Sequence: Synthetic primer
tttgttgtccagccaccatgatg

SEQ ID NO: 39 - DNA, 23 bp, Artificial Sequence
  Description of Artificial Sequence: Synthetic primer
tttgttgtccagccaccatgaaa

SEQ ID NO: 40 - DNA, 23 bp, Artificial Sequence
  Description of Artificial Sequence: Synthetic primer
tttgttgtccagccaccatgaca

SEQ ID NO: 41 - DNA, 23 bp, Artificial Sequence
  Description of Artificial Sequence: Synthetic primer
tttgttgtccagccaccatgaga

SEQ ID NO: 42 - DNA, 23 bp, Artificial Sequence
  Description of Artificial Sequence: Synthetic primer
tttgttgtccagccaccatgcta

SEQ ID NO: 43 - DNA, 23 bp, Artificial Sequence
  Description of Artificial Sequence: Synthetic primer
tttgttgtccagccaccatggta

SEQ ID NO: 44 - DNA, 23 bp, Artificial Sequence
  Description of Artificial Sequence: Synthetic primer
tttgttgtccagccaccatgtta

SEQ ID NO: 45 - DNA, 23 bp, Artificial Sequence
  Description of Artificial Sequence: Synthetic primer
tttgttgtccagccaccatcata

SEQ ID NO: 46 - DNA, 23 bp, Artificial Sequence
  Description of Artificial Sequence: Synthetic primer
tttgttgtccagccaccattata

SEQ ID NO: 47 - DNA, 23 bp, Artificial Sequence
  Description of Artificial Sequence: Synthetic primer
tttgttgtccagccaccataata

SEQ ID NO: 48 - DNA, 25 bp, Artificial Sequence
  Description of Artificial Sequence: Synthetic primer
  modified_base (23)..(23): N6-tert-butyl-benzyl-deoxyadenine
gttttgttgtccagccaccatgata

SEQ ID NO: 49 - DNA, 24 bp, Artificial Sequence
  Description of Artificial Sequence: Synthetic primer
  modified_base (22)..(22): N6-tert-butyl-benzyl-deoxyadenine
ttgtgttgtccagccaccatgata

SEQ ID NO: 50 - DNA, 30 bp, Artificial Sequence
  Description of Artificial Sequence: Synthetic primer
agtatttcatgaaacaaatgaatgatgcgt

SEQ ID NO: 51 - DNA, 30 bp, Artificial Sequence
  Description of Artificial Sequence: Synthetic primer
agtatttcatgaaacaaatgaatgatgctt

SEQ ID NO: 52 - DNA, 30 bp, Artificial Sequence
  Description of Artificial Sequence: Synthetic primer
agtatttcatgaaacaaatgaatgatggat

SEQ ID NO: 53 - DNA, 30 bp, Artificial Sequence
  Description of Artificial Sequence: Synthetic primer
agtatttcatgaaacaaatgaatgatgtat

SEQ ID NO: 54 - DNA, 30 bp, Artificial Sequence
  Description of Artificial Sequence: Synthetic primer
agtatttcatgaaacaaatgaatgatgaat

SEQ ID NO: 55 - DNA, 30 bp, Artificial Sequence
  Description of Artificial Sequence: Synthetic primer
  modified_base (29)..(29): N6-tert-butyl-benzyl-deoxyadenine
agtgtttcatgaaacaaatgaatgatgcat

SEQ ID NO: 56 - DNA, 30 bp, Artificial Sequence; identical to SEQ ID NO: 55
  Description of Artificial Sequence: Synthetic primer
  modified_base (28)..(28): N4-tert-butyl-benzyl-deoxycytosine

SEQ ID NO: 57 - DNA, 30 bp, Artificial Sequence; identical to SEQ ID NO: 50
  Description of Artificial Sequence: Synthetic primer
  modified_base (28)..(28): N4-ethyl-deoxycytosine

SEQ ID NO: 58 - DNA, 30 bp, Artificial Sequence
  Description of Artificial Sequence: Synthetic primer
  modified_base (29)..(29): N6-tert-butyl-benzyl-deoxyadenine
agtatttcatgaaacaaatgaatgattcat

SEQ ID NO: 59 - DNA, 30 bp, Artificial Sequence; identical to SEQ ID NO: 51
  Description of Artificial Sequence: Synthetic primer
  modified_base (28)..(28): N4-ethyl-deoxycytosine

SEQ ID NO: 60 - DNA, 30 bp, Artificial Sequence
  Description of Artificial Sequence: Synthetic primer
ataaaaattctttgtgcaacctacgtgaat

SEQ ID NO: 61 - DNA, 30 bp, Artificial Sequence
  Description of Artificial Sequence: Synthetic primer
ataaaaattctttgtgcaacctacgtgaaa

SEQ ID NO: 62 - DNA, 30 bp, Artificial Sequence
  Description of Artificial Sequence: Synthetic primer
ataaaaattctttgtgcaacctacgtgaca

SEQ ID NO: 63 - DNA, 30 bp, Artificial Sequence
  Description of Artificial Sequence: Synthetic primer
ataaaaattctttgtgcaacctacgtgaga

SEQ ID NO: 64 - DNA, 30 bp, Artificial Sequence
  Description of Artificial Sequence: Synthetic primer
ataaaaattctttgtgcaacctacgtgata

SEQ ID NO: 65 - DNA, 30 bp, Artificial Sequence
  Description of Artificial Sequence: Synthetic primer
ataaaaattctttgtgcaacctacgtgcaa

SEQ ID NO: 66 - DNA, 30 bp, Artificial Sequence
  Description of Artificial Sequence: Synthetic primer
ataaaaattctttgtgcaacctacgtggaa

SEQ ID NO: 67 - DNA, 30 bp, Artificial Sequence
  Description of Artificial Sequence: Synthetic primer
ataaaaattctttgtgcaacctacgtgtaa

SEQ ID NO: 68 - DNA, 30 bp, Artificial Sequence
  Description of Artificial Sequence: Synthetic primer
ataaaaattctttgtgcaacctacgtcaaa

SEQ ID NO: 69 - DNA, 30 bp, Artificial Sequence
  Description of Artificial Sequence: Synthetic primer
ataaaaattctttgtgcaacctacgttaaa

SEQ ID NO: 70 - DNA, 30 bp, Artificial Sequence
  Description of Artificial Sequence: Synthetic primer
ataaaaattctttgtgcaacctacgtaaaa

SEQ ID NO: 71 - DNA, 30 bp, Artificial Sequence
  Description of Artificial Sequence: Synthetic primer
atagaaattctttgtgcaacctacgtgaaa

SEQ ID NO: 72 - DNA, 30 bp, Artificial Sequence
  Description of Artificial Sequence: Synthetic primer
  modified_base (30)..(30): N6-tert-butyl-benzyl-deoxyadenine
atgaaaattctttgtgcaacctacgtgaaa

SEQ ID NO: 73 - DNA, 30 bp, Artificial Sequence; identical to SEQ ID NO: 72
  Description of Artificial Sequence: Synthetic primer
  modified_base (29)..(29): N6-tert-butyl-benzyl-deoxyadenine

SEQ ID NO: 74 - DNA, 30 bp, Artificial Sequence; identical to SEQ ID NO: 72
  Description of Artificial Sequence: Synthetic primer
  modified_base (28)..(28): N6-tert-butyl-benzyl-deoxyadenine

SEQ ID NO: 75 - DNA, 30 bp, Artificial Sequence; identical to SEQ ID NO: 62
  Description of Artificial Sequence: Synthetic primer
  modified_base (29)..(29): N4-tert-butyl-benzyl-deoxycytosine

SEQ ID NO: 76 - DNA, 30 bp, Artificial Sequence; identical to SEQ ID NO: 65
  Description of Artificial Sequence: Synthetic primer
  modified_base (28)..(28): N4-tert-butyl-benzyl-deoxycytosine

SEQ ID NO: 77 - DNA, 30 bp, Artificial Sequence; identical to SEQ ID NO: 62
  Description of Artificial Sequence: Synthetic primer
  modified_base (29)..(29): N4-methyl-deoxycytosine

SEQ ID NO: 78 - DNA, 30 bp, Artificial Sequence
  Description of Artificial Sequence: Synthetic primer
  modified_base (30)..(30): N6-tert-butyl-benzyl-deoxyadenine
ataaaaattctttgtgcaacctacgggaaa

SEQ ID NO: 79 - DNA, 30 bp, Artificial Sequence; identical to SEQ ID NO: 68
  Description of Artificial Sequence: Synthetic primer
  modified_base (27)..(27): N4-tert-butyl-benzyl-deoxycytosine

SEQ ID NO: 80 - DNA, 30 bp, Artificial Sequence; identical to SEQ ID NO: 78
  Description of Artificial Sequence: Synthetic primer
  modified_base (29)..(29): N6-tert-butyl-benzyl-deoxyadenine

SEQ ID NO: 81 - DNA, 30 bp, Artificial Sequence; identical to SEQ ID NO: 68
  Description of Artificial Sequence: Synthetic primer
  modified_base (27)..(27): N4-methyl-deoxycytosine

SEQ ID NO: 82 - DNA, 30 bp, Artificial Sequence; identical to SEQ ID NO: 68
  Description of Artificial Sequence: Synthetic primer
  modified_base (27)..(27): N4-methyl-deoxycytosine
  modified_base (30)..(30): N6-tert-butyl-benzyl-deoxyadenine

SEQ ID NO: 83 - DNA, 27 bp, Artificial Sequence
  Description of Artificial Sequence: Synthetic primer
caatttctacacgagatcctctctctg

SEQ ID NO: 84 - DNA, 27 bp, Artificial Sequence
  Description of Artificial Sequence: Synthetic primer
caatttctacacgagatcctctctcta

SEQ ID NO: 85 - DNA, 27 bp, Artificial Sequence
  Description of Artificial Sequence: Synthetic primer
caatttctacacgagatcctctctcaa

SEQ ID NO: 86 - DNA, 27 bp, Artificial Sequence
  Description of Artificial Sequence: Synthetic primer
caatttctacacgagatcctctctcca

SEQ ID NO: 87 - DNA, 27 bp, Artificial Sequence
  Description of Artificial Sequence: Synthetic primer
caatttctacacgagatcctctctcga

SEQ ID NO: 88 - DNA, 27 bp, Artificial Sequence
  Description of Artificial Sequence: Synthetic primer
caatttctacacgagatcctctctgta

SEQ ID NO: 89 - DNA, 27 bp, Artificial Sequence
  Description of Artificial Sequence: Synthetic primer
caatttctacacgagatcctctcttta

SEQ ID NO: 90 - DNA, 27 bp, Artificial Sequence
  Description of Artificial Sequence: Synthetic primer
caatttctacacgagatcctctctata

SEQ ID NO: 91 - DNA, 27 bp, Artificial Sequence
  Description of Artificial Sequence: Synthetic primer
caatttctacacgagatcctctcacta

SEQ ID NO: 92 - DNA, 27 bp, Artificial Sequence
  Description of Artificial Sequence: Synthetic primer
caatttctacacgagatcctctcccta

SEQ ID NO: 93 - DNA, 27 bp, Artificial Sequence
  Description of Artificial Sequence: Synthetic primer
caatttctacacgagatcctctcgcta

SEQ ID NO: 94 - DNA, 27 bp, Artificial Sequence
  Description of Artificial Sequence: Synthetic primer
cagtttctacacgagatcctctctcta

SEQ ID NO: 95 - DNA, 31 bp, Artificial Sequence
  Description of Artificial Sequence: Synthetic primer
  modified_base (31)..(31): N6-tert-butyl-benzyl-deoxyadenine
gaagcaatttctacacgagatcctctctcta

SEQ ID NO: 96 - DNA, 31 bp, Artificial Sequence; identical to SEQ ID NO: 95
  Description of Artificial Sequence: Synthetic primer
  modified_base (29)..(29): N4-tert-butyl-benzyl-deoxycytosine

SEQ ID NO: 97 - DNA, 27 bp, Artificial Sequence
  Description of Artificial Sequence: Synthetic primer
  modified_base (25)..(25): N4-tert-butyl-benzyl-deoxycytosine
cagtttctacacgagatcctctctcta

SEQ ID NO: 98 - DNA, 25 bp, Artificial Sequence
  Description of Artificial Sequence: Synthetic primer
gagatcctctctctgaaatcactga

SEQ ID NO: 99 - DNA, 25 bp, Artificial Sequence
  Description of Artificial Sequence: Synthetic primer
gagatcctctctctgaaatcactgc

SEQ ID NO: 100 - DNA, 25 bp, Artificial Sequence
  Description of Artificial Sequence: Synthetic primer
gagatcctctctctgaaatcactcc

SEQ ID NO: 101 - DNA, 25 bp, Artificial Sequence
  Description of Artificial Sequence: Synthetic primer
gagatcctctctctgaaatcacttc

SEQ ID NO: 102 - DNA, 25 bp, Artificial Sequence
  Description of Artificial Sequence: Synthetic primer
gagatcctctctctgaaatcactac

SEQ ID NO: 103 - DNA, 25 bp, Artificial Sequence
  Description of Artificial Sequence: Synthetic primer
gagatcctctctctgaaatcacagc

SEQ ID NO: 104 - DNA, 25 bp, Artificial Sequence
  Description of Artificial Sequence: Synthetic primer
gagatcctctctctgaaatcaccgc

SEQ ID NO: 105 - DNA, 25 bp, Artificial Sequence
  Description of Artificial Sequence: Synthetic primer
gagatcctctctctgaaatcacggc

SEQ ID NO: 106 - DNA, 25 bp, Artificial Sequence
  Description of Artificial Sequence: Synthetic primer
gagatcctctctctgaaatcagtgc

SEQ ID NO: 107 - DNA, 25 bp, Artificial Sequence
  Description of Artificial Sequence: Synthetic primer
gagatcctctctctgaaatcattgc

SEQ ID NO: 108 - DNA, 25 bp, Artificial Sequence
  Description of Artificial Sequence: Synthetic primer
gagatcctctctctgaaatcaatgc

SEQ ID NO: 109 - DNA, 25 bp, Artificial Sequence
  Description of Artificial Sequence: Synthetic primer
gggatcctctctctgaaatcactgc

SEQ ID NO: 110 - DNA, 25 bp, Artificial Sequence
  Description of Artificial Sequence: Synthetic primer
  modified_base (22)..(22): N4-tert-butyl-benzyl-deoxycytosine
gggatcctctctctgaaatcactgc

SEQ ID NO: 111 - DNA, 25 bp, Artificial Sequence
  Description of Artificial Sequence: Synthetic primer
  modified_base (25)..(25): N4-tert-butyl-benzyl-deoxycytosine
gggatcctctctctgaaatcactgc

SEQ ID NO: 112 - DNA, 25 bp, Artificial Sequence
  Description of Artificial Sequence: Synthetic primer
  modified_base (25)..(25): N4-tert-butyl-benzyl-deoxycytosine
gagatcctctctctgaaatcgctgc

SEQ ID NO: 113 - DNA, 25 bp, Artificial Sequence
  Description of Artificial Sequence: Synthetic primer
  modified_base (25)..(25): N4-tert-butyl-benzyl-deoxycytosine
gagatcctctctctgaaatcattgc

SEQ ID NO: 114 - DNA, 25 bp, Artificial Sequence
  Description of Artificial Sequence: Synthetic primer
  modified_base (24)..(24): N4-tert-butyl-benzyl-deoxycytosine
gagatcctctctctgaaatcactcc

SEQ ID NO: 115 - DNA, 25 bp, Artificial Sequence
  Description of Artificial Sequence: Synthetic primer
  modified_base (21)..(21): N6-tert-butyl-benzyl-deoxyadenine
  modified_base (24)..(24): Gclamp
gagatcctctctctgaaatcactcc

SEQ ID NO: 116 - DNA, 25 bp, Artificial Sequence
  Description of Artificial Sequence: Synthetic primer
  modified_base (23)..(23): N4-tert-butyl-benzyl-deoxycytosine
gagatcctctctctgaaatcaccgc

SEQ ID NO: 117 - DNA, 25 bp, Artificial Sequence
  Description of Artificial Sequence: Synthetic primer
  modified_base (24)..(24): N6-tert-butyl-benzyl-deoxyadenine
gagatcctctctctgaaatcactac

SEQ ID NO: 118 - DNA, 25 bp, Artificial Sequence
  Description of Artificial Sequence: Synthetic primer
  modified_base (23)..(23): N4-ethyl-deoxycytosine
gagatcctctctctgaaatcgccgc

SEQ ID NO: 119 - DNA, 25 bp, Artificial Sequence
  Description of Artificial Sequence: Synthetic primer
  modified_base (25)..(25): N4-tert-butyl-benzyl-deoxycytosine
gagatcctctctctgaaatcaccgc

SEQ ID NO: 120 - DNA, 25 bp, Artificial Sequence
  Description of Artificial Sequence: Synthetic primer
  modified_base (25)..(25): N4-tert-butyl-benzyl-deoxycytosine
gagatcctctctctgaaatcacggc

SEQ ID NO: 121 - DNA, 25 bp, Artificial Sequence
  Description of Artificial Sequence: Synthetic primer
  modified_base (22)..(22): N4-ethyl-deoxycytosine
  modified_base (24)..(24): N4-ethyl-deoxycytosine
gagatcctctctctgaaatcactcc

SEQ ID NO: 122 - DNA, 25 bp, Artificial Sequence
  Description of Artificial Sequence: Synthetic primer
  modified_base (22)..(22): N4-ethyl-deoxycytosine
gagatcctctctctgaaatcacggc

SEQ ID NO: 123 - DNA, 25 bp, Artificial Sequence
  Description of Artificial Sequence: Synthetic primer
  modified_base (22)..(22): N4-tert-butyl-benzyl-deoxycytosine
gagatcctctctctgaaatcaccgc

SEQ ID NO: 124 - DNA, 25 bp, Artificial Sequence
  Description of Artificial Sequence: Synthetic primer
  modified_base (23)..(23): N6-tert-butyl-benzyl-deoxyadenine
gagatcctctctctgaaatcacagc

SEQ ID NO: 125 - DNA, 25 bp, Artificial Sequence
  Description of Artificial Sequence: Synthetic primer
gagatcctctctctgaaatcactga

SEQ ID NO: 126 - DNA, 25 bp, Artificial Sequence
  Description of Artificial Sequence: Synthetic primer
gagatcctctctctgaaatcactgg

SEQ ID NO: 127 - DNA, 25 bp, Artificial Sequence
  Description of Artificial Sequence: Synthetic primer
gagatcctctctctgaaatcactcg

SEQ ID NO: 128 - DNA, 25 bp, Artificial Sequence
  Description of Artificial Sequence: Synthetic primer
gagatcctctctctgaaatcactag

SEQ ID NO: 129 - DNA, 25 bp, Artificial Sequence
  Description of Artificial Sequence: Synthetic primer
gagatcctctctctgaaatcacttg

SEQ ID NO: 130 - DNA, 25 bp, Artificial Sequence
  Description of Artificial Sequence: Synthetic primer
gagatcctctctctgaaatcacagg

SEQ ID NO: 131 - DNA, 25 bp, Artificial Sequence
  Description of Artificial Sequence: Synthetic primer
gagatcctctctctgaaatcaccgg

SEQ ID NO: 132 - DNA, 25 bp, Artificial Sequence
  Description of Artificial Sequence: Synthetic primer
gagatcctctctctgaaatcacggg

SEQ ID NO: 133 - DNA, 25 bp, Artificial Sequence
  Description of Artificial Sequence: Synthetic primer
gagatcctctctctgaaatcagtgg

SEQ ID NO: 134 - DNA, 25 bp, Artificial Sequence
  Description of Artificial Sequence: Synthetic primer
gagatcctctctctgaaatcaatgg

SEQ ID NO: 135 - DNA, 25 bp, Artificial Sequence
  Description of Artificial Sequence: Synthetic primer
gagatcctctctctgaaatcattgg

SEQ ID NO: 136 - DNA, 25 bp, Artificial Sequence
  Description of Artificial Sequence: Synthetic primer
gggatcctctctctgaaatcactgg

SEQ ID NO: 137 - DNA, 25 bp, Artificial Sequence
  Description of Artificial Sequence: Synthetic primer
  modified_base (22)..(22): N4-tert-butyl-benzyl-deoxycytosine
gggatcctctctctgaaatcactgg

SEQ ID NO: 138 - DNA, 25 bp, Artificial Sequence
  Description of Artificial Sequence: Synthetic primer
  modified_base (24)..(24): N4-tert-butyl-benzyl-deoxycytosine
gagatcctctctctgaaatcactcg

SEQ ID NO: 139 - DNA, 25 bp, Artificial Sequence
  Description of Artificial Sequence: Synthetic primer
  modified_base (21)..(21): N6-tert-butyl-benzyl-deoxyadenine
  modified_base (24)..(24): N4-ethyl-deoxycytosine
gagatcctctctctgaaatcactcg

SEQ ID NO: 140 - DNA, 25 bp, Artificial Sequence
  Description of Artificial Sequence: Synthetic primer
  modified_base (21)..(21): N6-tert-butyl-benzyl-deoxyadenine
gagatcctctctctgaaatcacttg

SEQ ID NO: 141 - DNA, 25 bp, Artificial Sequence
  Description of Artificial Sequence: Synthetic primer
  modified_base (24)..(24): N6-tert-butyl-benzyl-deoxyadenine
gagatcctctctctgaaatcactag

SEQ ID NO: 142 - DNA, 25 bp, Artificial Sequence
  Description of Artificial Sequence: Synthetic primer
  modified_base (24)..(24): N4-ethyl-deoxycytosine
gagatcctctctctgaaatcactcg

SEQ ID NO: 143 - DNA, 25 bp, Artificial Sequence
  Description of Artificial Sequence: Synthetic primer
  modified_base (22)..(22): N6-tert-butyl-benzyl-deoxyadenine
gagatcctctctctgaaatcaatgg

SEQ ID NO: 144 - DNA, 31 bp, Artificial Sequence
  Description of Artificial Sequence: Synthetic primer
  modified_base (21)..(21): Inosine
  modified_base (28)..(28): N4-tert-butyl-benzyl-deoxycytosine
ctatacgagatcctctctctnaaatcactgg

SEQ ID NO: 145 - DNA, 24 bp, Artificial Sequence
  Description of Artificial Sequence: Synthetic primer
agatcctctctctgaaatcactag

SEQ ID NO: 146 - DNA, 24 bp, Artificial Sequence
  Description of Artificial Sequence: Synthetic primer
agatcctctctctgaaatcacggg

SEQ ID NO: 147 - DNA, 26 bp, Artificial Sequence
  Description of Artificial Sequence: Synthetic primer
acgagatcctctctctgaaatcactg

SEQ ID NO: 148 - DNA, 26 bp, Artificial Sequence
  Description of Artificial Sequence: Synthetic primer
acgagatcctctctctgaaatcacta

SEQ ID NO: 149 - DNA, 26 bp, Artificial Sequence
  Description of Artificial Sequence: Synthetic primer
acgagatcctctctctgaaatcacaa

SEQ ID NO: 150 - DNA, 26 bp, Artificial Sequence
  Description of Artificial Sequence: Synthetic primer
acgagatcctctctctgaaatcacca

SEQ ID NO: 151 - DNA, 26 bp, Artificial Sequence
  Description of Artificial Sequence: Synthetic primer
acgagatcctctctctgaaatcacga

SEQ ID NO: 152 - DNA, 26 bp, Artificial Sequence
  Description of Artificial Sequence: Synthetic primer
acgagatcctctctctgaaatcagta

SEQ ID NO: 153 - DNA, 26 bp, Artificial Sequence
  Description of Artificial Sequence: Synthetic primer
acgagatcctctctctgaaatcaata

SEQ ID NO: 154 - DNA, 26 bp, Artificial Sequence
  Description of Artificial Sequence: Synthetic primer
acgagatcctctctctgaaatcatta

SEQ ID NO: 155 - DNA, 26 bp, Artificial Sequence
  Description of Artificial Sequence: Synthetic primer
acgagatcctctctctgaaatcccta

SEQ ID NO: 156 - DNA, 26 bp, Artificial Sequence
  Description of Artificial Sequence: Synthetic primer
acgagatcctctctctgaaatcgcta

SEQ ID NO: 157 - DNA, 26 bp, Artificial Sequence
  Description of Artificial Sequence: Synthetic primer
acgagatcctctctctgaaatctcta

SEQ ID NO: 158 - DNA, 26 bp, Artificial Sequence
  Description of Artificial Sequence: Synthetic primer
aggagatcctctctctgaaatcacta

SEQ ID NO: 159 - DNA, 26 bp, Artificial Sequence
  Description of Artificial Sequence: Synthetic primer
  modified_base (26)..(26): N6-tert-butyl-benzyl-deoxyadenine
aggagatcctctctctgaaatcacta

SEQ ID NO: 160 - DNA, 26 bp, Artificial Sequence
  Description of Artificial Sequence: Synthetic primer
  modified_base (24)..(24): N4-ethyl-deoxycytosine
aggagatcctctctctgaaatcacta

SEQ ID NO: 161 - DNA, 26 bp, Artificial Sequence
  Description of Artificial Sequence: Synthetic primer
  modified_base (23)..(23): N6-tert-butyl-benzyl-deoxyadenine
aggagatcctctctctgaaatcacta

SEQ ID NO: 162 - DNA, 26 bp, Artificial Sequence
  Description of Artificial Sequence: Synthetic primer
  modified_base (24)..(24): N6-tert-butyl-benzyl-deoxyadenine
acgagatcctctctctgaaatcaata

SEQ ID NO: 163 - DNA, 26 bp, Artificial Sequence
  Description of Artificial Sequence: Synthetic primer
  modified_base (25)..(25): N6-tert-butyl-benzyl-deoxyadenine
acgagatcctctctctgaaatcacaa

SEQ ID NO: 164 - DNA, 26 bp, Artificial Sequence
  Description of Artificial Sequence: Synthetic primer
  modified_base (22)..(22): N4-methyl-deoxycytosine
  modified_base (24)..(24): N6-tert-butyl-benzyl-deoxyadenine
acgagatcctctctctgaaatcaata

SEQ ID NO: 165 - DNA, 26 bp, Artificial Sequence
  Description of Artificial Sequence: Synthetic primer
  modified_base (25)..(25): N4-tert-butyl-benzyl-deoxycytosine
acgagatcctctctctgaaatcacca

SEQ ID NO: 166 - DNA, 26 bp, Artificial Sequence
  Description of Artificial Sequence: Synthetic primer
  modified_base (23)..(23): N4-tert-butyl-benzyl-deoxycytosine
acgagatcctctctctgaaatcccta

SEQ ID NO: 167 - DNA, 26 bp, Artificial Sequence
  Description of Artificial Sequence: Synthetic primer
agatcctctctctgaaatcactgagc

SEQ ID NO: 168 - DNA, 26 bp, Artificial Sequence
  Description of Artificial Sequence: Synthetic primer
agatcctctctctgaaatcactgaga

SEQ ID NO: 169 - DNA, 26 bp, Artificial Sequence
  Description of Artificial Sequence: Synthetic primer
agatcctctctctgaaatcactgaca

SEQ ID NO: 170 - DNA, 26 bp, Artificial Sequence
  Description of Artificial Sequence: Synthetic primer
agatcctctctctgaaatcactgaaa

SEQ ID NO: 171 - DNA, 26 bp, Artificial Sequence
  Description of Artificial Sequence: Synthetic primer
agatcctctctctgaaatcactgata

SEQ ID NO: 172 - DNA, 26 bp, Artificial Sequence
  Description of Artificial Sequence: Synthetic primer
agatcctctctctgaaatcactgcga

SEQ ID NO: 173 - DNA, 26 bp, Artificial Sequence
  Description of Artificial Sequence: Synthetic primer
agatcctctctctgaaatcactgtga

SEQ ID NO: 174 - DNA, 26 bp, Artificial Sequence
  Description of Artificial Sequence: Synthetic primer
agatcctctctctgaaatcactggga

SEQ ID NO: 175 - DNA, 26 bp, Artificial Sequence
  Description of Artificial Sequence: Synthetic primer
agatcctctctctgaaatcactcaga

SEQ ID NO: 176 - DNA, 26 bp, Artificial Sequence
  Description of Artificial Sequence: Synthetic primer
agatcctctctctgaaatcactaaga

SEQ ID NO: 177 - DNA, 26 bp, Artificial Sequence
  Description of Artificial Sequence: Synthetic primer
agatcctctctctgaaatcacttaga

SEQ ID NO: 178 - DNA, 26 bp, Artificial Sequence
  Description of Artificial Sequence: Synthetic primer
aggtcctctctctgaaatcactgaga

SEQ ID NO: 179 - DNA, 27 bp, Artificial Sequence
  Description of Artificial Sequence: Synthetic primer
  modified_base (27)..(27): N6-tert-butyl-benzyl-deoxyadenine
gaggtcctctctctgaaatcactgaga

SEQ ID NO: 180 - DNA, 27 bp, Artificial Sequence
  Description of Artificial Sequence: Synthetic primer
  modified_base (25)..(25): N6-tert-butyl-benzyl-deoxyadenine
gaggtcctctctctgaaatcactgaga

SEQ ID NO: 181 - DNA, 27 bp, Artificial Sequence
  Description of Artificial Sequence: Synthetic primer
gagatcctctctctgaaatcactgaaa

SEQ ID NO: 182 - DNA, 27 bp, Artificial Sequence
  Description of Artificial Sequence: Synthetic primer
gagatcctctctctgaaatcactggga

SEQ ID NO: 183 - DNA, 27 bp, Artificial Sequence
  Description of Artificial Sequence: Synthetic primer
gagatcctctctctgaaatcactaaga

SEQ ID NO: 184 - DNA, 24 bp, Artificial Sequence
  Description of Artificial Sequence: Synthetic primer
atcctctctctgaaatcactgagc

SEQ ID NO: 185 - DNA, 24 bp, Artificial Sequence
  Description of Artificial Sequence: Synthetic primer
atcctctctctgaaatcactgagg

SEQ ID NO: 186 - DNA, 24 bp, Artificial Sequence
  Description of Artificial Sequence: Synthetic primer
atcctctctctgaaatcactgaag

SEQ ID NO: 187 - DNA, 24 bp, Artificial Sequence
  Description of Artificial Sequence: Synthetic primer
atcctctctctgaaatcactgacg

SEQ ID NO: 188 - DNA, 24 bp, Artificial Sequence
  Description of Artificial Sequence: Synthetic primer
atcctctctctgaaatcactgatg

SEQ ID NO: 189 - DNA, 24 bp, Artificial Sequence
  Description of Artificial Sequence: Synthetic primer
atcctctctctgaaatcactgcgg

SEQ ID NO: 190 - DNA, 24 bp, Artificial Sequence
  Description of Artificial Sequence: Synthetic primer
atcctctctctgaaatcactgggg

SEQ ID NO: 191 - DNA, 24 bp, Artificial Sequence
  Description of Artificial Sequence: Synthetic primer
atcctctctctgaaatcactgtgg

SEQ ID NO: 192 - DNA, 24 bp, Artificial Sequence
  Description of Artificial Sequence: Synthetic primer
atcctctctctgaaatcactaagg

SEQ ID NO: 193 - DNA, 24 bp, Artificial Sequence
  Description of Artificial Sequence: Synthetic primer
atcctctctctgaaatcactcagg

SEQ ID NO: 194 - DNA, 24 bp, Artificial Sequence
  Description of Artificial Sequence: Synthetic primer
atcctctctctgaaatcacttagg

SEQ ID NO: 195 - DNA, 24 bp, Artificial Sequence
  Description of Artificial Sequence: Synthetic primer
tcctctctctgaaatcactgagca

SEQ ID NO: 196 - skipped

SEQ ID NO: 197 - DNA, 24 bp, Artificial Sequence
  Description of Artificial Sequence: Synthetic primer
tcctctctctgaaatcactgagct

SEQ ID NO: 198 - DNA, 24 bp, Artificial Sequence
  Description of Artificial Sequence: Synthetic primer
tcctctctctgaaatcactgagat

SEQ ID NO: 199 - DNA, 24 bp, Artificial Sequence
  Description of Artificial Sequence: Synthetic primer
tcctctctctgaaatcactgaggt

SEQ ID NO: 200 - DNA, 24 bp, Artificial Sequence
  Description of Artificial Sequence: Synthetic primer
tcctctctctgaaatcactgagtt

SEQ ID NO: 201 - DNA, 24 bp, Artificial Sequence
  Description of Artificial Sequence: Synthetic primer
tcctctctctgaaatcactgaact

SEQ ID NO: 202 - DNA, 24 bp, Artificial Sequence
  Description of Artificial Sequence: Synthetic primer
tcctctctctgaaatcactgacct

SEQ ID NO: 203 - DNA, 24 bp, Artificial Sequence
  Description of Artificial Sequence: Synthetic primer
tcctctctctgaaatcactgatct

SEQ ID NO: 204 - DNA, 24 bp, Artificial Sequence
  Description of Artificial Sequence: Synthetic primer
tcctctctctgaaatcactgcgct

SEQ ID NO: 205 - DNA, 24 bp, Artificial Sequence
  Description of Artificial Sequence: Synthetic primer
tcctctctctgaaatcactgggct

SEQ ID NO: 206 - DNA, 24 bp, Artificial Sequence
  Description of Artificial Sequence: Synthetic primer
tcctctctctgaaatcactgtgct

SEQ ID NO: 207 - DNA, 29 bp, Artificial Sequence
  Description of Artificial Sequence: Synthetic primer
catgaaacaaatgaatgatgcacatcatg

SEQ ID NO: 208 - DNA, 29 bp, Artificial Sequence
  Description of Artificial Sequence: Synthetic primer
catgaaacaaatgaatgatgcacatcatc

SEQ ID NO: 209 - DNA, 29 bp, Artificial Sequence
  Description of Artificial Sequence: Synthetic primer
catgaaacaaatgaatgatgcacatcaac

SEQ ID NO: 210 - DNA, 29 bp, Artificial Sequence
  Description of Artificial Sequence: Synthetic primer
catgaaacaaatgaatgatgcacatcacc

SEQ ID NO: 211 - DNA, 29 bp, Artificial Sequence
  Description of Artificial Sequence: Synthetic primer
catgaaacaaatgaatgatgcacatcagc

SEQ ID NO: 212 - DNA, 29 bp, Artificial Sequence
  Description of Artificial Sequence: Synthetic primer
catgaaacaaatgaatgatgcacatcctc

SEQ ID NO: 213 - DNA, 29 bp, Artificial Sequence
  Description of Artificial Sequence: Synthetic primer
catgaaacaaatgaatgatgcacatcgtc

SEQ ID NO: 214 - DNA, 29 bp, Artificial Sequence
  Description of Artificial Sequence: Synthetic primer
catgaaacaaatgaatgatgcacatcttc

SEQ ID NO: 215 - DNA, 29 bp, Artificial Sequence
  Description of Artificial Sequence: Synthetic primer
catgaaacaaatgaatgatgcacataatc

SEQ ID NO: 216 - DNA, 29 bp, Artificial Sequence
  Description of Artificial Sequence: Synthetic primer
catgaaacaaatgaatgatgcacatgatc

SEQ ID NO: 217 - DNA, 29 bp, Artificial Sequence
  Description of Artificial Sequence: Synthetic primer
catgaaacaaatgaatgatgcacattatc

SEQ ID NO: 218 - DNA, 23 bp, Artificial Sequence
  Description of Artificial Sequence: Synthetic primer
agccaccatgatgtgcatcattc

SEQ ID NO: 219 - DNA, 23 bp, Artificial Sequence
  Description of Artificial Sequence: Synthetic primer
agccaccatgatgtgcatcatta

SEQ ID NO: 220 - DNA, 23 bp, Artificial Sequence
  Description of Artificial Sequence: Synthetic primer
agccaccatgatgtgcatcataa

SEQ ID NO: 221 - DNA, 23 bp, Artificial Sequence
  Description of Artificial Sequence: Synthetic primer
agccaccatgatgtgcatcatga

SEQ ID NO: 222 - DNA, 23 bp, Artificial Sequence
  Description of Artificial Sequence: Synthetic primer
agccaccatgatgtgcatcaata

SEQ ID NO: 223 - DNA, 23 bp, Artificial Sequence
  Description of Artificial Sequence: Synthetic primer
agccaccatgatgtgcatcacta

SEQ ID NO: 224 - DNA, 23 bp, Artificial Sequence
  Description of Artificial Sequence: Synthetic primer
agccaccatgatgtgcatcagta

SEQ ID NO: 225 - DNA, 23 bp, Artificial Sequence
  Description of Artificial Sequence: Synthetic primer
agccaccatgatgtgcatcctta

SEQ ID NO: 226 - DNA, 23 bp, Artificial Sequence
  Description of Artificial Sequence: Synthetic primer
agccaccatgatgtgcatcgtta

SEQ ID NO: 227 - DNA, 23 bp, Artificial Sequence
  Description of Artificial Sequence: Synthetic primer
agccaccatgatgtgcatcttta

SEQ ID NO: 228 - DNA, 23 bp, Artificial Sequence
  Description of Artificial Sequence: Synthetic primer
aggcaccatgatgtgcatcatta

SEQ ID NO: 229 - DNA, 23 bp, Artificial Sequence
  Description of Artificial Sequence: Synthetic primer
  modified_base (23)..(23): N6-tert-butyl-benzyl-deoxyadenine
aggcaccatgatgtgcatcatta

SEQ ID NO: 230 - DNA, 23 bp, Artificial Sequence
  Description of Artificial Sequence: Synthetic primer
  modified_base (20)..(20): N6-tert-butyl-benzyl-deoxyadenine
aggcaccatgatgtgcatcatta